(19) United States
(12) Patent Application Publication
(10) Pub. No.: US 2023/0298700 A1
(43) Pub. Date: Sep. 21, 2023

(54) DEVICE FOR PREDICTING MUTATION OF VIRUS, METHOD FOR PREDICTING MUTATION OF VIRUS, AND PROGRAM

(71) Applicant: Tohoku University, Sendai-shi, Miyagi (JP)

(72) Inventor: Koetsu OGASAWARA, Sendai-shi, Miyagi (JP)

(21) Appl. No.: 18/017,039

(22) PCT Filed: Jul. 21, 2021
(86) PCT No.: PCT/JP2021/027331

§ 371 Date: Jan. 19, 2023

(30) Foreign Application Priority Data
Jul. 22, 2020 (JP) 2020-125563

(51) Int. Cl. G16B 30/10; G16B 40/00; G06N 20/00

(52) U.S. Cl. CPC G16B 30/10; G16B 40/00; G06N 20/00

(57) ABSTRACT

A viral mutation prediction device has an acquisition unit which acquires gene sequence data of a genome of a virus, an extraction unit which extracts C (cytosine) or G (guanine) from the acquired gene sequence data of the genome and extracts contexts in which a mutation from C or G to U (uracil) occurs or has occurred, a separation unit which checks whether there is an amino acid mutation when C or G has changed to U and which separates sequences with the amino acid mutation as nonsynonymous substitutions and separates sequences without the amino acid mutation as synonymous substitutions, a learning unit which learns using the sequence data of the synonymous substitutions for learning data and a prediction unit which predicts a mutation of the virus using the learned results.

### TECHNICAL FIELD

The present invention relates to a viral mutation prediction device, a viral mutation prediction method and a program.

The present application claims priority from patent application No. 2020-125563 filed in Japan on Jul. 22, 2020, and the contents thereof are incorporated here by reference.

### BACKGROUND ART

Viruses are characterized by the inability of self-proliferation and can proliferate using other cells. That is, viruses use various enzymes such as a host polymerase for proliferation. It is known that there are DNA viruses and RNA viruses. DNA viruses proliferate by synthesizing messenger RNA of the viral genome DNA using a host RNA polymerase and synthesizing protein. DNA viruses are known to have fewer gene mutations than RNA viruses because DNA viruses have a mechanism of correcting a DNA replication error introduced in the process of proliferation.

It is known that many mutations are introduced into RNA viruses to change the viruses as the infection spreads, as it can be typically seen with influenza. That is, RNA viruses have more gene mutations than DNA viruses. For example, coronaviruses such as the novel coronavirus (SARS-CoV-2) and SARS are also RNA viruses, and mutations have been observed. Coronaviruses, however, have RNA-proofreading enzymes in their viral genomes, and thus large-scale gene deletion and substitutions and mutations of several bases are not easily caused. Accordingly, it is known that coronaviruses have many point mutations. Here, a point mutation is a change due to deletion, substitution or insertion of a base.

A host RNA editing enzyme is known to be involved in a point mutation of an RNA virus. For mutations of the novel coronavirus, it is suggested that point mutations are caused by RNA editing enzymes, ADARs, APOBECs and the like. For point mutations of RNA viruses, results suggesting the involvement of especially ADARs have been presented. In addition, the base sequence of −2 to +2 has been suggested to be characteristic for a point mutation of an RNA virus, where the mutation site by an RNA editing enzyme is 0 and the two 5′-end bases and the two 3′-end bases of the surrounding base sequence are represented by −2 and +2, respectively (for example, see NPL 1).

Prediction of mutations in influenza viruses has been started so far, regarding prediction of mutations in viruses, and mutations are predicted using the hemagglutinin (HA) structure as an indicator. However, prediction of mutations in viruses having RNA-proofreading enzymes, such as the novel coronavirus, has not been conducted.

### CITATION LIST

### Non Patent Literature

NPL 1: Di Giorgio, S., et al. Evidence for host-dependent RNA editing in the transcriptome of SARS-CoV-2. Science Advances: eabb5813, 2020.

### SUMMARY OF INVENTION

### Technical Problem

RNA viruses such as the novel coronavirus undergo mutations. When a virus mutates, the antibody tests and the antigen tests used for diagnoses which have been produced before the viral mutation become ineffective, and the therapeutic agents are no longer effective. Viral mutations have problems because the locations of the mutations on the genome and the substituted bases can be identified only after the mutations occur. In order to produce an antibody test or an antigen test kit, it has been required to first identify the mutation sites after the mutations have been introduced and to newly create the protein used for the antibody test or the antigen test. Accordingly, it takes a lot of time to produce a diagnostic agent or a therapeutic agent for new mutations.

The invention has been made considering the above problems, and an object thereof is to provide a viral mutation prediction device, a viral mutation prediction method and a program which can predict a viral mutation in advance before the mutation occurs.

### Solution to Problem

The invention includes the following aspects.

[1] A viral mutation prediction device, having an acquisition unit which acquires gene sequence data of a genome of a virus, an extraction unit which extracts C (cytosine) or G (guanine) from the acquired gene sequence data of the genome and extracts contexts in which a mutation from C or G to U (uracil) occurs or has occurred, a separation unit which checks whether there is an amino acid mutation when C or G has changed to U and which separates sequences with the amino acid mutation as nonsynonymous substitutions and separates sequences without the amino acid mutation as synonymous substitutions, a learning unit which learns using the sequence data of the synonymous substitutions for learning data and a prediction unit which predicts a mutation of the virus using the learned results.

[2] A viral mutation prediction device, having an acquisition unit which acquires gene sequence data of a genome of a virus, an extraction unit which extracts C (cytosine), G (guanine), A (adenine), U (uracil) or T (thymine) from the acquired gene sequence data of the genome and extracts contexts in which a mutation from G to A, from A to G, from U to C or from T to C occurs or has occurred, a separation unit which checks whether there is an amino acid mutation when the base sequences of the extracted contexts have changed and which separates sequences with the amino acid mutation as nonsynonymous substitutions and separates sequences without the amino acid mutation as synonymous substitutions, a learning unit which learns using the sequence data of the synonymous substitutions for learning data and a prediction unit which predicts a mutation of the virus using the learned results.

[3] The viral mutation prediction device further has a sampling unit which selects a predetermined number of synonymous substitutions from the synonymous substitutions, and the learning unit uses the sequence data of the synonymous substitutions selected by the sampling unit for learning data.

[4] The viral mutation prediction device further has a feature value addition and selection unit which adds a feature value that is a value characterized by selecting two bases from the four kinds of RNA bases, A (adenine), U, G and C, and that is used for learning, and the learning unit also uses the feature value for learning data.

[5] In the viral mutation prediction device, the range of the contexts is −3 to +3 or more and −10 to +10 or less.

[6] In the viral mutation prediction device, the virus is SARS-CoV-2.

[7] A viral mutation prediction method in which an acquisition unit acquires gene sequence data of a genome of a virus, an extraction unit extracts C (cytosine) or G (guanine) from the acquired gene sequence data of the genome and extracts contexts in which a mutation from C or G to U (uracil) occurs or has occurred, a separation unit checks whether there is an amino acid mutation when C or G has changed to U, separates sequences with the amino acid mutation as nonsynonymous substitutions and separates sequences without the amino acid mutation as synonymous substitutions, a learning unit learns using the sequence data of the synonymous substitutions for learning data, and a prediction unit predicts a mutation of the virus using the learned results.

[8] A viral mutation prediction method in which an acquisition unit acquires gene sequence data of a genome of a virus, an extraction unit extracts C (cytosine), G (guanine), A (adenine), U (uracil) or T (thymine) from the acquired gene sequence data of the genome and extracts contexts in which a mutation from G to A, from A to G, from U to C or from T to C occurs or has occurred, a separation unit checks whether there is an amino acid mutation when the base sequences of the extracted contexts have changed, separates sequences with the amino acid mutation as nonsynonymous substitutions and separates sequences without the amino acid mutation as synonymous substitutions, a learning unit learns using the sequence data of the synonymous substitutions for learning data, and a prediction unit predicts a mutation of the virus using the learned results.

[9] A program which causes a computer to acquire gene sequence data of a genome of a virus, to extract C (cytosine) or G (guanine) from the acquired gene sequence data of the genome, to extract contexts in which a mutation from C or G to U (uracil) occurs or has occurred, to check in a separation unit whether there is an amino acid mutation when C or G has changed to U, to separate sequences with the amino acid mutation as nonsynonymous substitutions, to separate sequences without the amino acid mutation as synonymous substitutions, to learn using the sequence data of the synonymous substitutions for learning data and to predict a mutation of the virus using the learned results.

[10] A program which causes a computer to acquire gene sequence data of a genome of a virus,

to extract C (cytosine), G (guanine), A (adenine), U (uracil) or T (thymine) from the acquired gene sequence data of the genome, to extract contexts in which a mutation from G to A, from A to G, from U to C or from T to C occurs or has occurred, to check whether there is an amino acid mutation when the base sequences of the extracted contexts have changed, to separate sequences with the amino acid mutation as nonsynonymous substitutions, to separate sequences without the amino acid mutation as synonymous substitutions, to learn using the sequence data of the synonymous substitutions for learning data and to predict a mutation of the virus using the learned results.

### Advantageous Effects of Invention

According to the invention, a viral mutation can be predicted in advance before the mutation occurs.

### BRIEF DESCRIPTION OF DRAWINGS

FIG. 1 is a figure showing an example of the constitution of the viral mutation prediction device according to an embodiment.

FIG. 2 is a figure showing the distribution of point mutations in SARS-CoV-2 genomes.

FIG. 3 is a figure showing the counts of point mutations in genes.

FIG. 4 is a figure showing the point mutation rates per 100 bases in genes.

FIG. 5 is a figure showing the results of examination of mutated nucleic acid bases.

FIG. 6 is a figure showing the results of examining the bases which the respective bases have been mutated from.

FIG. 7 A figure showing the mutation patterns of genes.

FIG. 8 is a figure showing the counts of mutations obtained by dividing the counts of point mutations in genes by the gene lengths.

FIG. 9 is a figure showing the characteristics of the base sequences at both sides of C-to-U point mutations.

FIG. 10 is a figure showing the characteristics of the base sequences at both sides of G-to-A point mutations.

FIG. 11 is a figure showing the characteristics of the base sequences at both sides of A-to-G point mutations.

FIG. 12 is a figure showing the characteristics of the base sequences at both sides of U-to-C point mutations.

FIG. 13 is a figure showing the characteristics of the contexts that are three bases upstream and downstream of mutations from C to U (n=2401).

FIG. 14 is a figure showing the increases or the decreases [%] from the expectation values corresponding to the bases in the contexts of all the C residues in SARS-CoV-2 sequences.

FIG. 15 is a figure showing the proportions of the contexts of all the cytosine residues in the unmasked region of a reference sequence.

FIG. 16 is a flowchart of the learning procedures of the viral mutation prediction device according to an embodiment.

FIG. 17 is an image of mapping and mutation records.

FIG. 18 is a figure showing example combinations of two positions for a case using synonymous substitutions (without an amino acid mutation).

FIG. 19 is a figure showing an example of the top 30 selected feature values.

FIG. 20 is a figure showing an example relation between the context and the score of a case without the addition of feature values and without selection.

FIG. 21 is a figure showing an example relation between the context and the score of a case with the addition of feature values and with selection.

FIG. 22 is a figure showing the average scores of each context and each regularization parameter of a case with the addition of feature values and with selection.

FIG. 23 is a figure showing the standard deviations of the scores of each context and each regularization parameter of a case with the addition of feature values and with selection.

FIG. 24 is a flowchart of the processing procedures of mutation prediction according to an embodiment.

FIG. 25 is a figure showing an example of the information displayed on an image display device during mutation prediction.

FIG. 26 is a figure showing example results of calculation by logistic regression.

FIG. 27 is a figure showing mutation records and mutation prediction.

FIG. 28 is a figure showing a phylogenetic tree.

FIG. 29 is a figure showing the mutation sites on the genomes of selected four mutated forms and the locations of the RNA sequences used for a pseudo-infection model.

FIG. 30 is a figure showing the TNF-α production induced by ssRNAs.

FIG. 31 is a figure showing the IL-6 production induced by ssRNAs.

FIG. 32 is a figure showing example processing contents and example processing procedures of the analysis program according to an embodiment.

FIG. 33 is a figure showing example hyperparameter values of models which were optimized by grid search for each base sequence range.

FIG. 34 is a figure showing the coefficients of a regression equation for the base sequence range of −10 to +10 on a histogram.

FIG. 35 is a figure showing the coefficients of a regression equation for the base sequence range of −10 to +10 on a histogram.

FIG. 36 is a figure showing the coefficients of a regression equation for the base sequence range of −10 to +10 on a histogram.

FIG. 37 is a figure showing a box plot of a histogram of the coefficients of a regression equation for the base sequence range of −10 to +10.

FIG. 38 is a figure showing the summary and the characteristics of the compared learning models.

FIG. 39 is a figure showing example results of analysis of the summary statistics of the AUC scores of each model.

FIG. 40 is a figure showing example AUC scores before processing.

FIG. 41 is a figure showing example AUC scores after processing.

FIG. 42 is a figure showing the ROC curves of models for the base sequence range of −2 to +2 and the first cross-validation round.

FIG. 43 is a figure showing the ROC curves of models for the base sequence range of −2 to +2 and the second cross-validation round.

FIG. 44 is a figure showing an example method for splitting learning data by five cross-validation rounds.

FIG. 45 is a figure for explaining the method for measuring the generalization performance.

FIG. 46 is a box plot showing the base sequence ranges and the learning models for mutations from G to U.

FIG. 47 is a box plot showing the base sequence ranges and the learning models for mutations from G to A (adenine).

FIG. 48 is a box plot showing the base sequence ranges and the learning models for mutations from A to G.

FIG. 49 is a box plot showing the base sequence ranges and the learning models for mutations from U to C (from T (thymine) to C).

### DESCRIPTION OF EMBODIMENTS

Embodiments of the invention are explained below referring to the figures. In the following embodiments, an example in which the subject virus is SARS-CoV-2 is explained.

[Outline of SARS-CoV-2 Virus]

Currently, vaccines, diagnostic methods and therapeutic methods for SARS-CoV-2 are required. Vaccines and antibody tests are produced based on the protein (or the gene sequence) of SARS-CoV-2. According to genomic analysis, there are some variants of SARS-CoV-2 which are classified into three types, A, B and C. As a result, it is necessary to collect mutated forms of SARS-CoV-2 for vaccines and antibody tests.

Although the SARS-CoV-2 variants contain some gene mutations, the influence of the mutations on infection is unknown. Mutations are introduced into viruses through errors during self-replication or by cell-derived RNA editing enzymes. RNA editing enzymes are known to cause mutations in RNA viruses.

RNA editing enzymes such as adenosine deaminases acting on RNA (ADARs) and apolipoprotein B mRNA editing enzyme, catalytic polypeptides (APOBECs) have been studied in RNA virus infections. ADAR is an enzyme which extracts the amino group from adenosine and which converts adenosine into inosine and has a function of acting primarily on double-stranded RNA. APOBECs, a family of cytidine deaminases, are enzymes which extract the amino group from cytidine and convert cytidine into uracil. APOBECs have been reported to function using ssDNA as a substrate. Moreover, APOBEC1, APOBEC3A and APOBEC3G also recognize ssRNA as a substrate. However, it remains unclear whether a mutation of a SARS-CoV-2 mutant is induced by host RNA editing.

Accordingly, in the embodiment, by focusing on RNA editing enzymes and searching the viral genome based on the characteristic sequences of several bases before and after gene mutations of the virus, sites which may be mutated in the future and the substituting bases are predicted. When a viral mutation can be predicted in advance, time for preparing a diagnostic agent or a therapeutic agent for a new mutation can be secured, and a diagnostic agent or a therapeutic agent can be applied soon after the mutation occurs.

[Example Constitution of Device for Predicting Point Mutation of Virus]

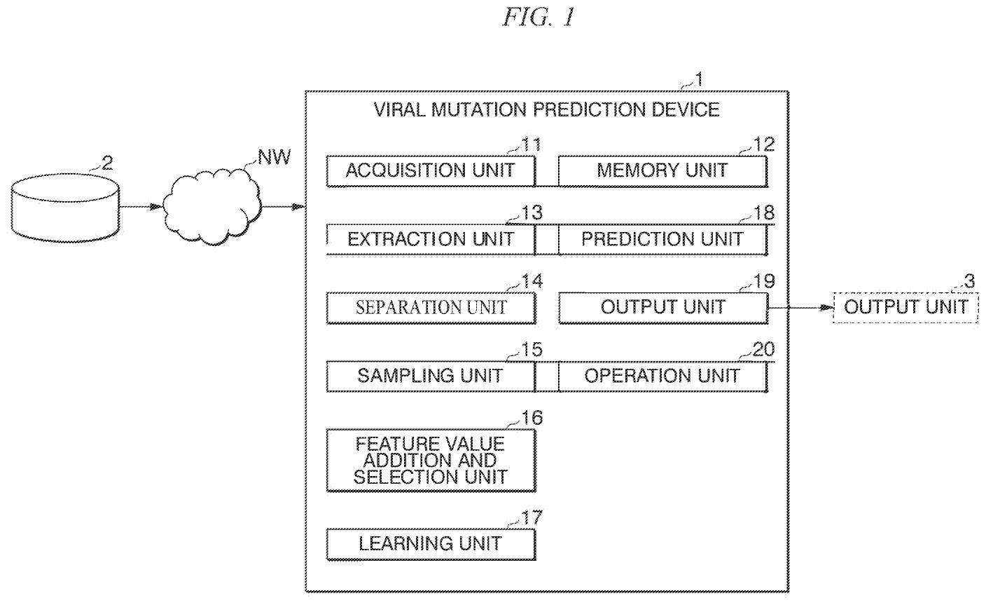

FIG. 1 is a figure showing an example of the constitution of a viral mutation prediction device 1 according to the embodiment. As shown in FIG. 1, the viral mutation prediction device 1 has an acquisition unit 11, a memory unit 12, an extraction unit 13, a separation unit 14, a sampling unit 15, a feature value addition and selection unit 16, a learning unit 17, a prediction unit 18, an output unit 19 and an operation unit 20.

The viral mutation prediction device 1 acquires data from a DB (database) 2 through a network NW. The viral mutation prediction device 1 predicts a mutation through learning of the characteristics of gene mutations from the acquired data.

The acquisition unit 11 is, for example, a wireless network circuit. The acquisition unit 11 acquires data from the DB 2 (for example, GISAID (Global initiative on sharing all influenza data; https://www.gisaid.org/)) through the network NW. The data are, for example, the gene sequences of the genomes of SARS-CoV-2 of the world and are plural.

The memory unit 12 memorizes the acquired genome data of SARS-CoV-2. The memory unit 12 memorizes the information showing whether a regularization parameter C has been mutated or not. When C (cytosine) or G (guanine) has changed to U (uracil), the memory unit 12 memorizes the results of checking whether there is an amino acid mutation. The memory unit 12 memorizes an algorithm, a program, a threshold and the like which are necessary for learning and prediction.

The extraction unit 13 extracts C from the acquired genomes of SARS-CoV-2. The extraction unit 13 also extracts contexts in which a mutation from C or G to U occurs or has occurred from the acquired genomes of SARS-CoV-2. Here, a context is a sequence set of several bases before and after the mutation site.

The separation unit 14 extracts mutation sites from C or G to U from the acquired genome data of SARS-CoV-2 and maps the extracted mutation sites on one genome. The separation unit 14 causes the memory unit 12 to memorize the information showing whether C or G has been mutated or not. When C or G has changed to U, the separation unit 14 checks whether there is an amino acid mutation and causes the memory unit 12 to memorize the checking results. When C or G has changed to U, the separation unit 14 checks whether there is an amino acid mutation, separates sequences with an amino acid mutation as nonsynonymous substitutions and separates sequences without an amino acid mutation as synonymous substitutions.

The sampling unit 15 selects a first predetermined number of sequences without an amino acid substitution (synonymous substitutions). To reduce noises, the sampling unit 15 selects a second predetermined number of sequences, which is fewer than the first predetermined number, from the first predetermined number of selected sequences as learning data. Here, the sampling does not have to be conducted. In this case, all the synonymous substitutions may be used for learning data. Moreover, the sampling unit 15 may also select the first predetermined number of sequences without an amino acid substitution (synonymous substitutions) and use the sequences as learning data.

The feature value addition and selection unit 16 adds a feature value (parameter). Here, the feature value will be described below. For example, the feature value is a value characterized by selecting two bases from the four kinds of RNA bases, A, U, G and C.

The learning unit 17 uses the second predetermined number of selected sequences as learning data and uses the rest of the first predetermined number as test data. The learning unit 17 conducts learning using the feature value and the learning data. In this regard, the learning unit 17 does not have to use the feature value for learning. Here, the learning unit 17 learns, for example, using an algorithm such as a neural network, a support vector machine, reinforcement learning and deep learning. Artificial intelligence (AI: Artificial Interigence) may be used for learning.

The prediction unit 18 predicts a point mutation using the learned results.

The output unit 19 displays information showing the results predicted by the prediction unit 18 on an image display device 3. Here, the image display device 3 may also be, for example, a tablet device or the like.

The operation unit 20 is, for example, a touch panel sensor provided on the image display device 3, a mouse or the like. The operation unit 20 detects the operation results operated by a user.

[Analysis Results of SARS-CoV-2]

Here, the results of analysis of SARS-CoV-2 conducted by the present inventor and the like are explained. The present inventor and the like comprehensively analyzed 7800 gene sequences of the genomes of SARS-CoV-2 of the world collected from GISAID. During the collection, overlapping sequences, sequences with unclear collection dates and the like were excluded. As a result, 7804 sequences were acquired from GISAID.

First, as a result of phylogenetic network analysis of the acquired sequences to create a phylogenetic tree, a frequency of 5000 point mutations or more was calculated.

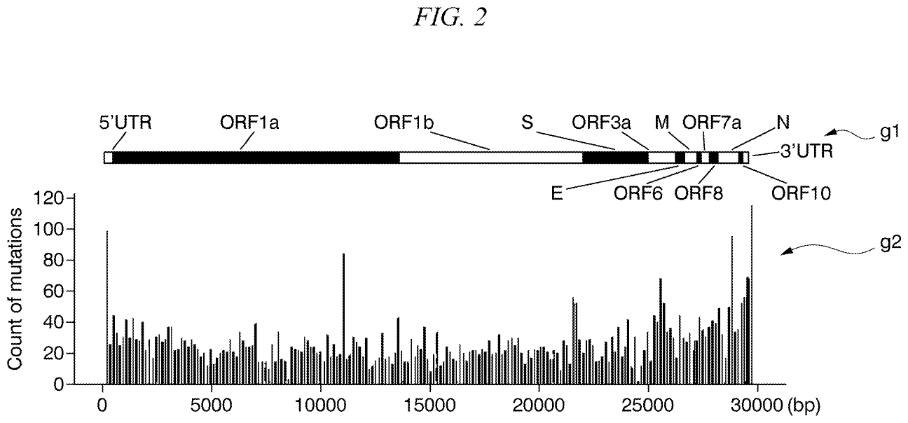

Next, the locations of the point mutations were analyzed. FIG. 2 is a figure showing the distribution of the point mutations in the SARS-CoV-2 genomes. Here, the upper figure in FIG. 2 is a figure (g1) showing the locations of genes in the full-length ssRNA. The lower histogram g2 of FIG. 2 shows the counts of mutations at the locations. In the histogram g2, the vertical axis is the count of mutations, and the horizontal axis is the base number (bp). As shown in FIG. 2, the average count of point mutations per 150 nucleotides (bin) was about 28, but higher frequencies of point mutations were observed at some locations.

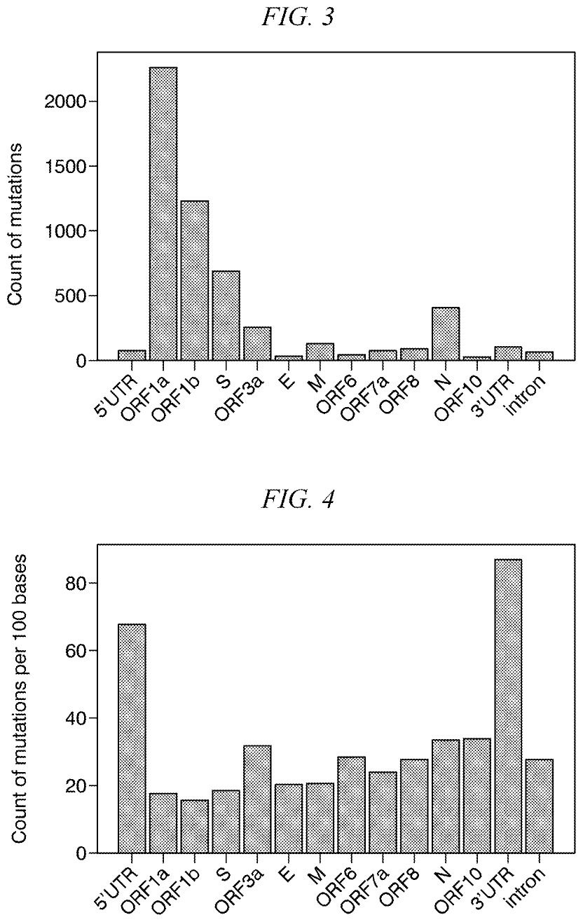

Next, to further analyze the bias of point mutations in the genes, the point mutations of each gene were counted. FIG. 3 is a figure showing the counts of point mutations in the genes. In FIG. 3, the horizontal axis shows the gene name, and the vertical axis is the count of mutations. As shown in FIG. 3, ORF-1a and ORF-1b included many point mutations.

However, more mutations may occur because ORF-1a and ORF-1b are much longer than other regions as shown in FIG. 2. Thus, the point mutation rates per 100 bases in the genes were estimated. FIG. 4 is a figure showing the point mutation rates per 100 bases in the genes. In FIG. 4, the horizontal axis shows the gene name, and the vertical axis is the point mutation rate per 100 bases. As shown in FIG. 4, after normalization by the gene length, the frequencies of point mutations in the 5′-untranslated region (UTR) and the 3′-UTR were the highest.

The results suggest that SARS-CoV-2 mutants have point mutations.

Next, the present inventor and the like visualized the gene mutations and thus analyzed the characteristics of the gene mutations.

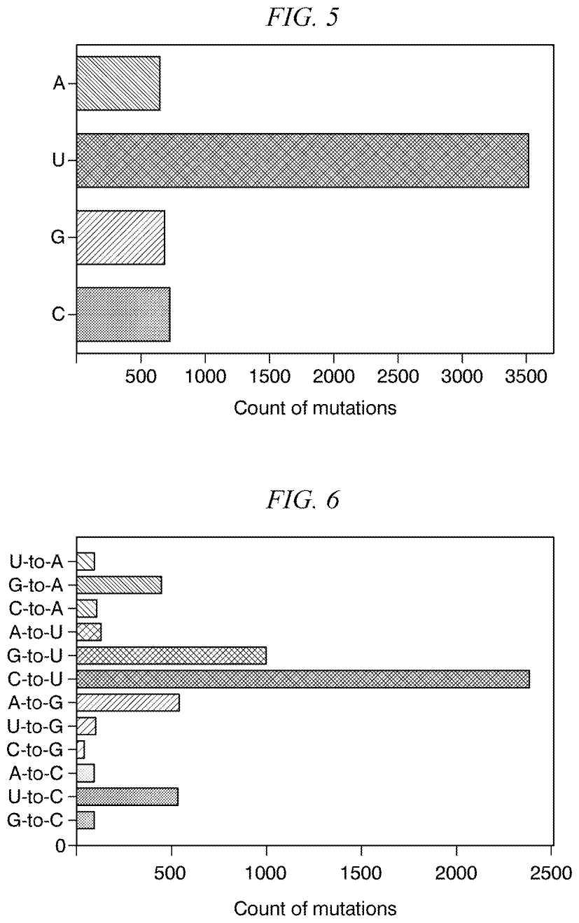

FIG. 5 is a figure showing the results of examination of mutated nucleic acid bases. The horizontal axis is the count of substituting bases after the point mutations, and the vertical axis shows the bases (A (adenine), U, G (guanine) and C). As shown in FIG. 5, it was found that the mutations to U account for a half or more.

FIG. 6 is a figure showing the results of examining the bases which the respective bases have been mutated from. The horizontal axis shows the counts of the original bases and the substituting bases for each point mutation, and the vertical axis is a base to a base. As a result, it was found that many of the mutations to U are mutations from C and G (especially C). Moreover, it was also found as follows: mutations to A are dominant in mutations from G; mutations to G are dominant in mutations from A; and mutations to C are dominant in mutations from U. Mutations from C to U and from G to A are known to be introduced by APOBECs, and mutations from A to G and from U to C are known to be introduced by ADARs. In the embodiment, for example, a mutation from C to U is also expressed by C-to-U.

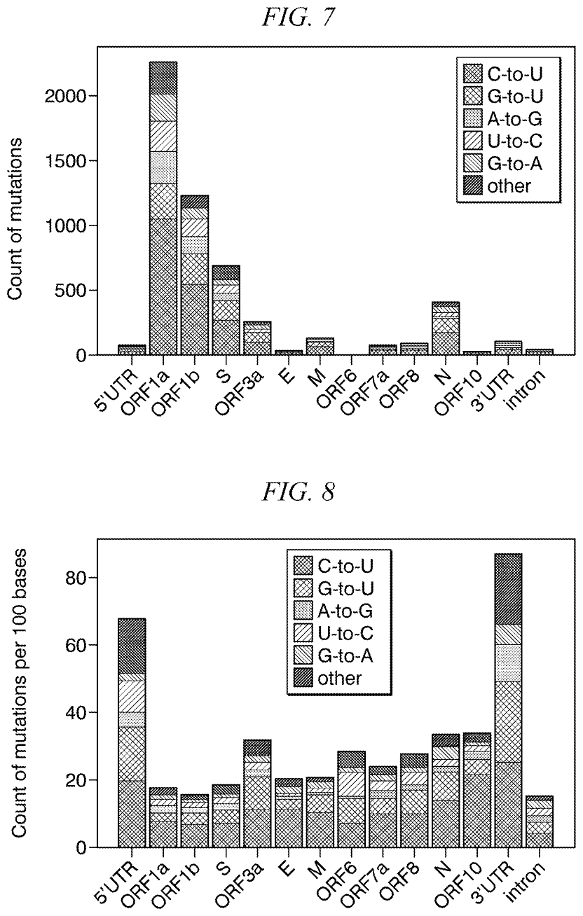

FIG. 7 is a figure showing the mutation patterns of genes. FIG. 8 is a figure showing the counts of mutations obtained by dividing the counts of point mutations of the genes by the gene lengths. In FIG. 7 and FIG. 8, the horizontal axis shows the gene name. The vertical axis in FIG. 7 is the count of mutations. The vertical axis in FIG. 8 is the count of mutations per 100 bases. From FIG. 7 and FIG. 8, C-to-U mutations were dominant although there were some differences among the genes.

Furthermore, of the mutations observed in FIG. 5 to FIG. 8, C-to-U and G-to-A are consistent with the mutations introduced by APOBECs, and A-to-G and C-to-U are consistent with the mutations introduced by ADARs. Thus, the present inventor and the like examined the contexts that are one base upstream and downstream of the four mutations.

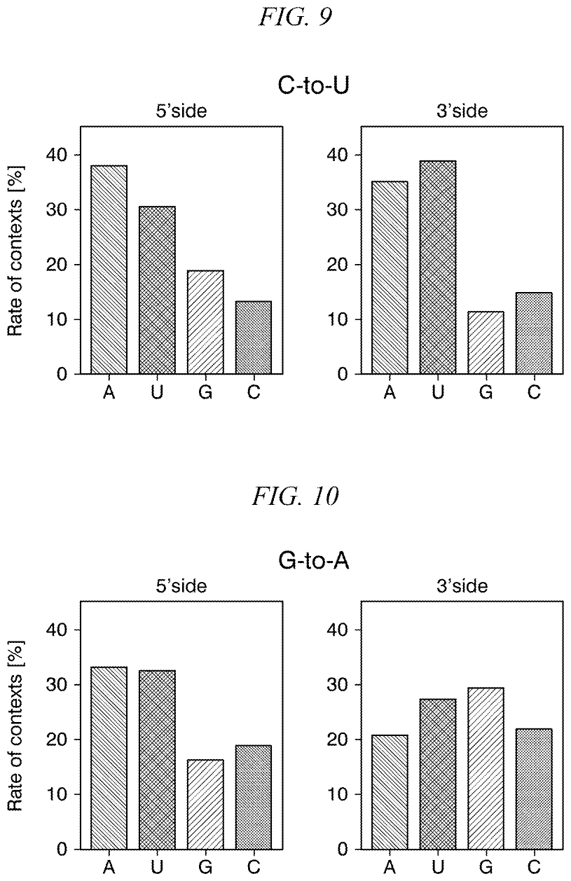

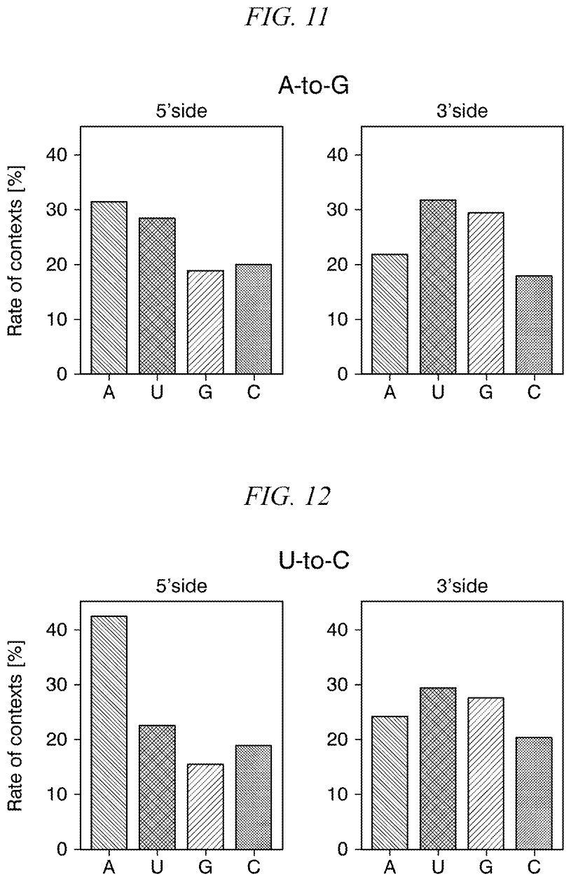

FIG. 9 is a figure showing the characteristics of the base sequences at both sides of C-to-U point mutations. FIG. 10 is a figure showing the characteristics of the base sequences at both sides of G-to-A point mutations. FIG. 11 is a figure showing the characteristics of the base sequences at both sides of A-to-G point mutations. FIG. 12 is a figure showing the characteristics of the base sequences at both sides of U-to-C point mutations. In FIG. 9 to FIG. 12, the horizontal axis shows the base name, and the vertical axis shows the proportions [%] of A, U, G and C. Moreover, in FIG. 9 to FIG. 12, the left graph shows the bases at the 5′ side (−1) of the mutation sites, and the right graph shows the bases at the 3′ side (−1) of the mutation sites.

As shown in FIG. 9, A and U were often adjacent to a C-to-U mutation both at the 5′ side and the 3′ side. As shown in FIG. 10, however, A and U were often adjacent to a G-to-A mutation at the 5′ side, and G and U were often adjacent at the 3′ side. The complementary sequences thereof are [C/U]C and C[A/U].

As shown in FIG. 11, A and U were often adjacent to an A-to-G mutation at the 5′ side, and U and G were often adjacent at the 3′ side. Moreover, as shown in FIG. 12, U was often adjacent to a U-to-C mutation at the 5′ side, and G and U were often adjacent at the 3′ side. The complementary sequences thereof are CA and [A/C]C.

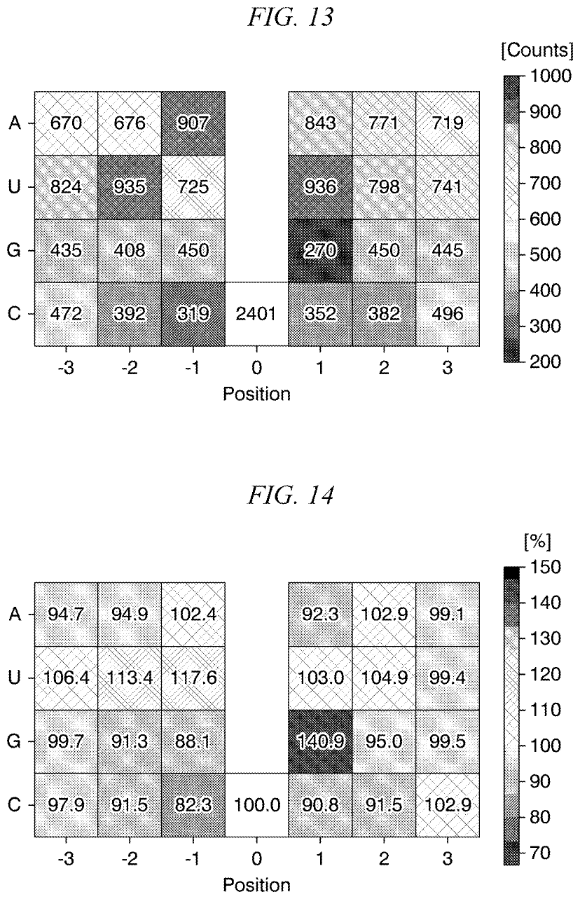

Next, the contexts that were three bases upstream and downstream of mutations from C to U (n=2401), which were most frequently observed, were examined in more detail, and the results are explained. FIG. 13 is a figure showing the characteristics of the contexts that are three bases upstream and downstream of mutations from C to U (n=2401). Here, 0 indicates the mutation site, and the negative numbers and the positive numbers indicate the upstream and downstream positions, respectively. In FIG. 13, the horizontal direction is the location of context. The values are the counts of the bases AUGC at the locations. As shown in FIG. 13, the counts of A and U residues were very high before and after substituted C. This is believed to be because SARS-CoV-2 is biased for many A and U residues (30% A and 32% U).

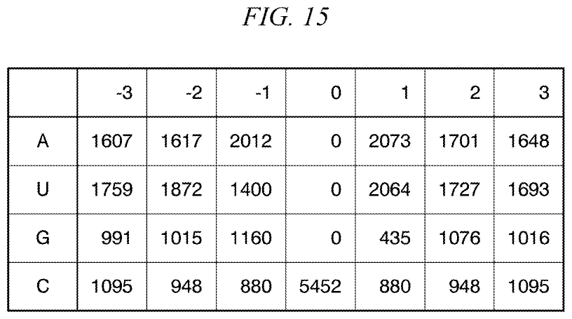

Because the characteristics in FIG. 13 were obtained, the contexts of all the C residues in the SARS-CoV-2 sequences were examined and used as the expectation values, and the increases or the decreases [%] of the bases from the corresponding expectation values were examined. FIG. 14 is a figure showing the increases or the decreases [%] from the expectation values corresponding to the respective bases in the contexts of all the C residues in the SARS-CoV-2 sequences. In FIG. 14, the horizontal direction is the location of context. As shown in FIG. 14, the value of U was high at positions+2 and +1, and the value of G was high at −1 (p<10∧−3, fisher exact test). At position +1, the value of C was low (p<0.01, fisher exact test). This may suggest the substrate specificity of APOBECs which introduce mutations. Here, the expectation values of the contexts at the upstream side (−3) and the downstream side (+3) of the cytosine residues in C-to-U mutations were calculated from the proportions of the contexts of all the cytosine residues in the unmasked region of a reference sequence (FIG. 15). FIG. 15 is a figure showing the proportions of the contexts of all the cytosine residues in the unmasked region of the reference sequence.

From the above analyses, the following four characteristics of gene mutations were found.
I. There are many uracil (U) mutations.
II. There are many mutations from cytosine (C) to uracil (U).
III. RNA editing enzymes are involved in gene mutations.
IV. There are characteristic sequences of one base to three bases before and after uracil mutations.

[Learning Procedures]

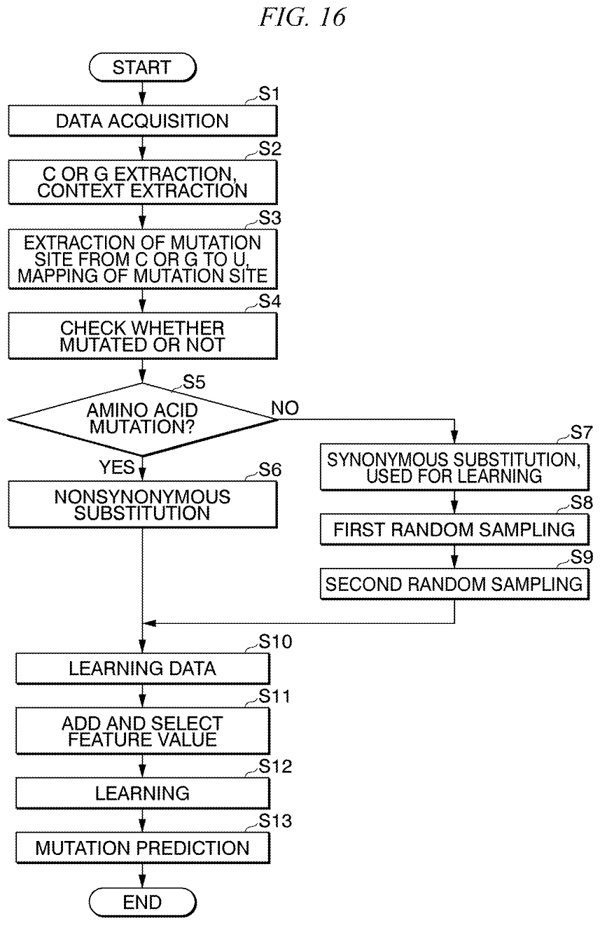

Next, example learning procedures of the viral mutation prediction device 1 are explained. Here, in the embodiment, genomes of SARS-CoV-2 were used as the teaching data. FIG. 16 is a flowchart of the learning procedures of the viral mutation prediction device 1 according to the embodiment.

(Step S1) The acquisition unit 11 acquires genome data of SARS-CoV-2 from the DB 2 (for example, GISAID). The acquisition unit 11 causes the memory unit 12 to memorize the acquired genome data of SARS-CoV-2.

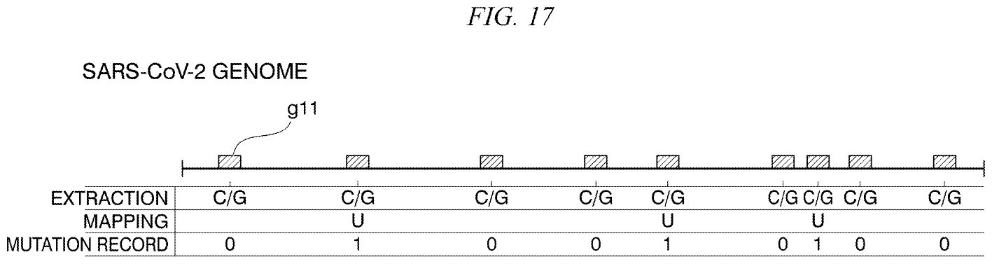

(Step S2) The extraction unit 13 selects C or G from the acquired genomes of SARS-CoV-2. The extraction unit 13 also extracts contexts g11 (FIG. 17) in which a mutation from C or G to U occurs or has occurred from the acquired genomes of SARS-CoV-2. FIG. 17 is an image of mapping and mutation records. Here, the contexts are, for example, of three kinds (−2 to +2, −3 to +3 and −10 to +10).

(Step S3) The separation unit 14 extracts the mutation sites from C or G to U from the acquired genome data of SARS-CoV-2 and maps the extracted mutation sites on one genome (FIG. 17).

(Step S4) The separation unit 14 causes the memory unit 12 to memorize the information showing whether C or G has been mutated or not (FIG. 17). For example, the separation unit 14 causes to memorize the mutations from C or G to U as 1 and memorize C or G without a mutation as 0 through numeric conversion.

(Step S5) When C or G has changed to U, the separation unit 14 checks whether there is an amino acid mutation and causes the memory unit 12 to memorize the checking results. When it is determined that there is an amino acid mutation (the step S5; YES), the separation unit 14 proceeds to the processing of the step S6. When it is determined that there is no amino acid mutation (step S5; NO), the separation unit 14 proceeds to the processing of the step S7.

(Step S6) The separation unit 14 determines that the mutation is a nonsynonymous substitution and also uses the data for learning.

(Step S7) The separation unit 14 determines that the mutation is a synonymous substitution and also uses the data for learning. Here, mutations were observed at 675 sites of about 1800 sites of synonymous substitutions. After the processing, the separation unit 14 proceeds to the processing of the step S8.

(Step S8) The sampling unit 15 selects 1000 sequences without an amino acid substitution (synonymous substitutions) (500 with a mutation and 500 without a mutation) (first random sampling). In this regard, the sampling unit 15 conducts the random selection five times and selects 1000 sequences without an amino acid substitution (synonymous substitutions).

(Step S9) In general, in machine learning, the learning data are often set at 60 to 80%, and thus the sampling unit 15 selects 800 of the selected 1000 sequences as the learning data (second random sampling). In this regard, the sampling unit 15 conducts the random selection five times and chooses 800 sequences. The sampling unit 15 does not have to conduct the processing.

(Step S10) The learning unit 17 uses the selected 800 sequences as the learning data and the remaining 200 sequences as the test data. Here, the learning unit 17 also uses those without a mutation for the learning data.

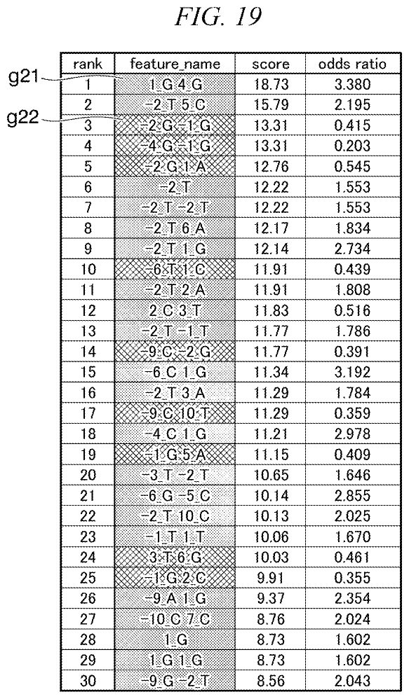

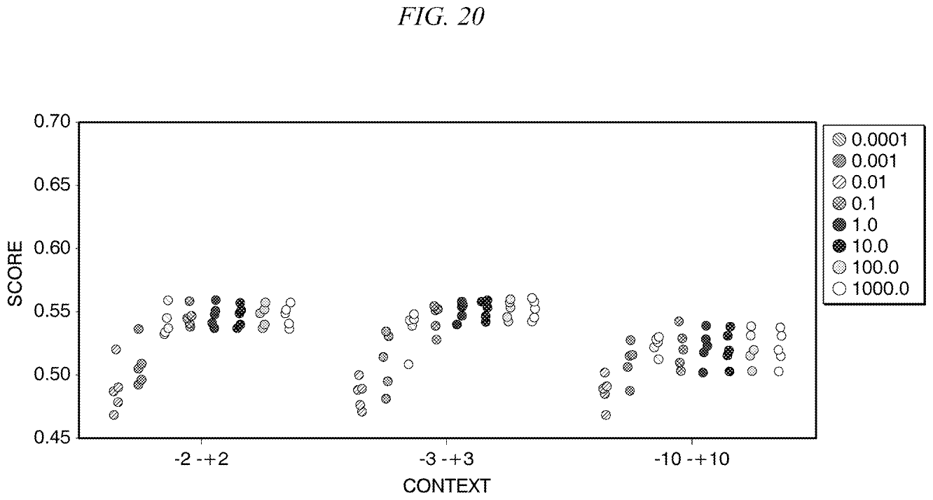

(Step S11) The feature value addition and selection unit 16 adds feature values (parameters). For example, in a sequence of −10 to +10 bases, there are four types of RNA bases, A, U, G and C, and the sequence has 20 bases. Thus, there are 80 types of feature value (=4×20). There are 6400 types, the square of 80, because two bases thereof are selected for characterization, and there are 3200 types for the feature value, namely the half thereof, because it is a combination. Subsequently, the feature value addition and selection unit 16 selects, for example, the top 30 from the 3200 types of parameter. Here, the number of feature values is an example and does not limit the invention. The feature value addition and selection unit 16 selected a chi-squared test for the standard and used SelectKBest (chi2, K=30). The feature values are used for improving the scores (the score here is synonymous with the percentage of correct answers) during learning. The feature values are combinations of two bases selected in the contexts as shown in FIG. 19.

(Step S12) The learning unit 17 conducts learning using the feature values and the learning data.

(Step S13) The prediction unit 18 predicts a point mutation using the learned results. The prediction will be described below

Although an example including three types of contexts (−2 to +2, −3 to +3 and −10 to +10) has been described above, the invention is not limited thereto. The contexts should be −3 to +3 or more and −10 to +10 or less. Here, −3 to +3 or more and −10 to +10 or less includes −4 to +4, . . . , −9 to +9.

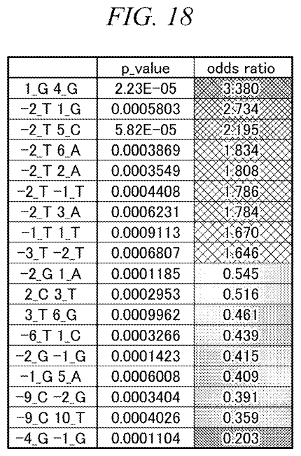

FIG. 18 is a figure showing example combinations of two positions for a case using synonymous substitutions (without an amino acid mutation). For example, in “1_G 4_G” at the first line, 1_G indicates G at position+1, and 4_G indicates G at position+4. Moreover, “−2_T 1_G” at line 2 shows the context TNCG.

FIG. 19 is a figure showing an example of the top 30 selected feature values. Here, the hatching g21 indicates increases, and the hatching g22 indicates decreases. The number of selected feature values is not limited to 30.

[Comparison of Scores Between Presence and Absence of Features]

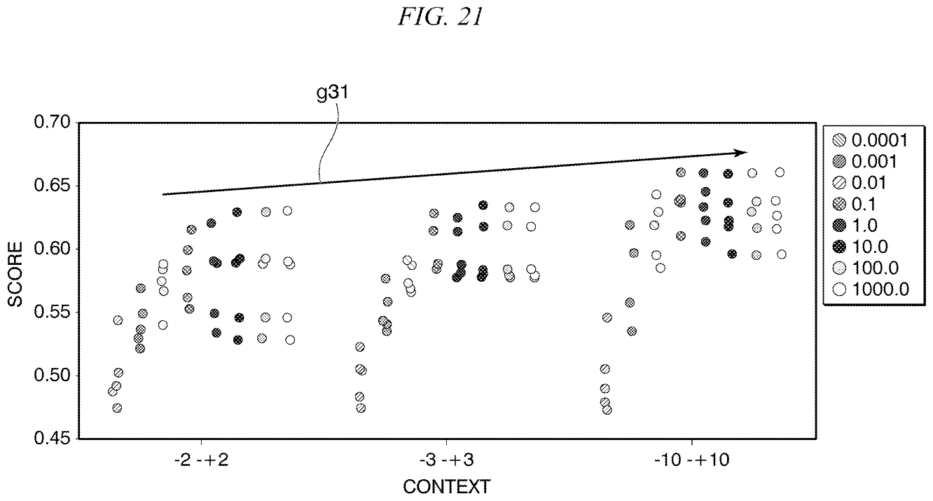

Here, a difference in the scores of the learning results between a case without the addition of feature values and a case with the addition is explained. Using 800 sites as the learning data and 200 sites as the test data of 1000 sites obtained by random sampling, cross-validation was conducted (n=5). The results are shown in FIG. 20 and FIG. 21.

FIG. 20 is a figure showing an example relation between the context and the score of a case without the addition of feature values and without selection. FIG. 21 is a figure showing an example relation between the context and the score of a case with the addition of feature values and with selection. In FIG. 20 and FIG. 21, the horizontal axis is the context {(−2, +2), (−3, +3), (−10, +10)}, and the vertical axis is the score. In each context, the dots are regularization parameter C values in logistic regression and are 0.0001, 0.001, 0.01, 0.1, 1.0, 10.0, 100.0 and 1000.0, respectively, from the left. One dot is an average score (n=5) of cross-validation. Here, the regularization parameters indicate that learning is easier as the value is larger. The scores indicate the percentages of correct answers, and the dispersion indicates the robustness against data bias.

In the case without the addition of feature values and without selection, the scores of the learning results did not improve even when the context range was increased as shown in FIG. 20. On the other hand, in the case with the addition of feature values and with selection, the scores of the learning results increased when the context range was increased as indicated with the arrow g31 in FIG. 21. It was thus found that the addition of feature values and selection are effective for mutation prediction.

In the embodiment, to predict a mutation through machine learning as described above, feature values are added, and 800 values are learned. At this point, the prediction unit 18 predicts through calculation by multiplying with a coefficient according to the order of the top 30 by adding feature values (top 30). The feature values (of the top 30) include truly important values and noises.

The C values were expressed as the easiness of learning, and this means that the C values were used for classification (a small C value means without noises, and a large value includes noises) because calculation was conducted by multiplying a coefficient based on the feature values which also included noises.

For example, C=0.0001 means incomplete learning because the learning does not include noises, and C=1000 means noises are included for learning.

In FIG. 21, regarding an appropriate C value (both for −3 to +3 and −10 to +10), the location at which the score first reached the highest point (C=0.1 or 1.0) is believed to be appropriate (the possibility that the calculation could be done with the true coefficient of feature value is high). Here, because overtraining includes noises, it is important to reduce noises as much as possible and use truly important feature values for learning.

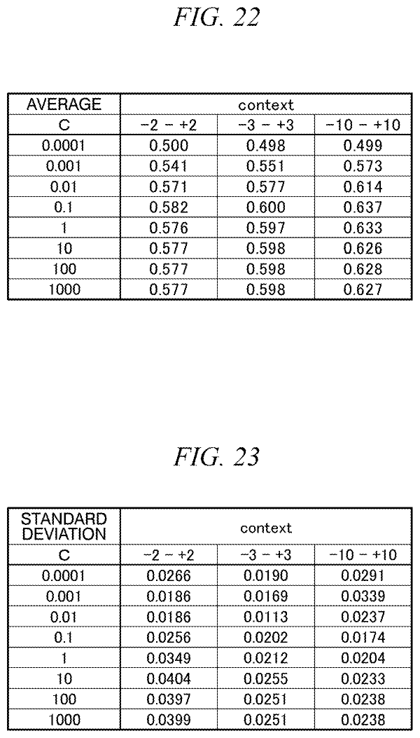

FIG. 22 is a figure showing the average scores of each context and each regularization parameter of a case with the addition of feature values and with selection. FIG. 23 is a figure showing the standard deviations of the scores of each context and each regularization parameter of a case with the addition of feature values and with selection.

As shown in FIG. 22 and FIG. 23, the comparison between the contexts of −2 to +2 and 3 to +3 shows that the scores of −3 to +3 are higher and that the dispersions (standard deviations) are smaller. Because a higher score shows a higher percentage of correct answers and because a smaller dispersion shows higher validity of the obtained results, it is believed to be practical.

Furthermore, the context of −10 to +10 had higher scores and smaller dispersions than −3 to +3. Accordingly, the context of −3 to +3 is better than −2 to +2, and −10 to +10 is better than −3 to +3. This means that the context of −10 to +10 was the best.

[Mutation Prediction]

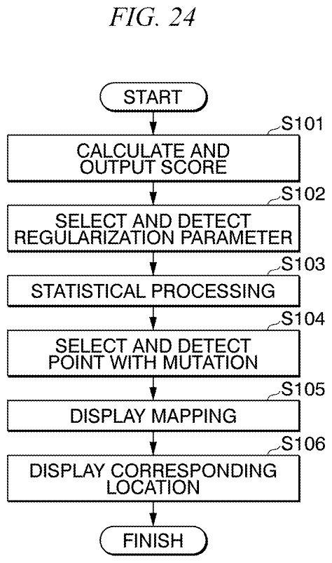

Next, an example of mutation prediction in the embodiment is explained. FIG. 24 is a flowchart of the processing procedures of mutation prediction according to the embodiment. Here, before the prediction, the learning described above is conducted in advance.

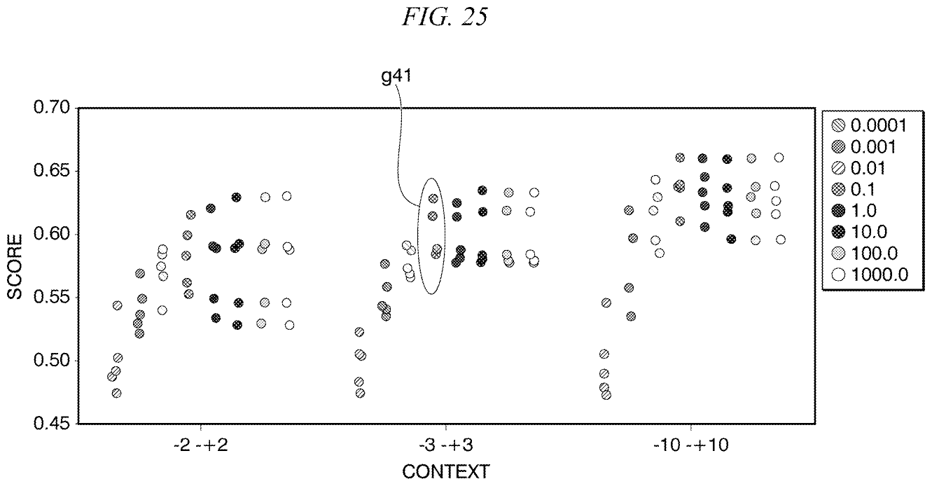

(Step S101) The prediction unit 18 calculates the scores of the predicted results and displays the calculated scores on the image display device 3 through the output unit 19. As a result, a graph showing the relation between the context and the score, such as the graph of FIG. 25 for example, is displayed on the image display device 3. FIG. 25 is a figure showing an example of the information displayed on the image display device 3 during mutation prediction.

(Step S102) The user sees the displayed image (FIG. 25) and selects, for example the region g41 of C=0.1 of the context of −3 to +3. The operation unit 20 outputs the selected information selected by the user to the prediction unit 18.

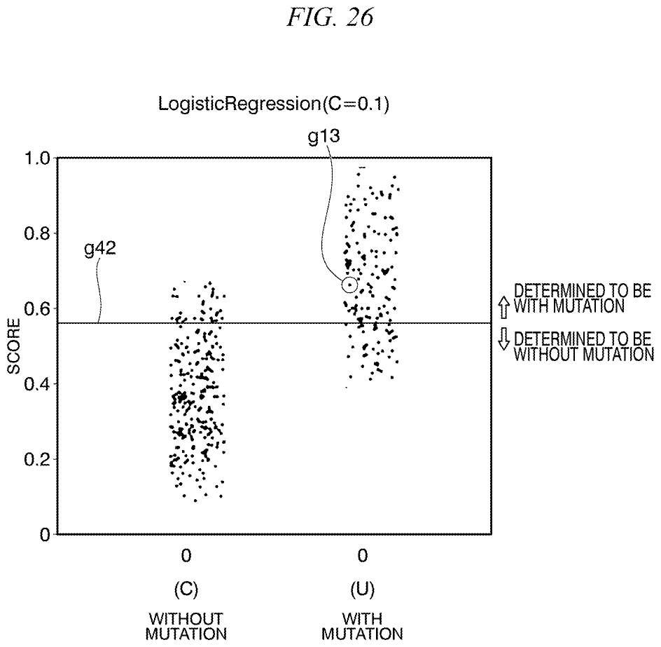

(Step S103) The prediction unit 18 conducts statistical processing such as one shown in FIG. 26 by a predetermined algorithm (for example, logistic regression) for the selected regularization parameter of the context. FIG. 26 is a figure showing example results of calculation by logistic regression. The vertical axis in FIG. 26 is the score, and the line g42 is the threshold for presence or absence of mutation. The prediction unit 18 displays a graph like the graph in FIG. 26 on the image display device 3.

(Step S104) The user sees the displayed image (FIG. 26) and selects a point with a mutation, for example, the point g43. The operation unit 20 outputs the selected information selected by the user to the prediction unit 18.

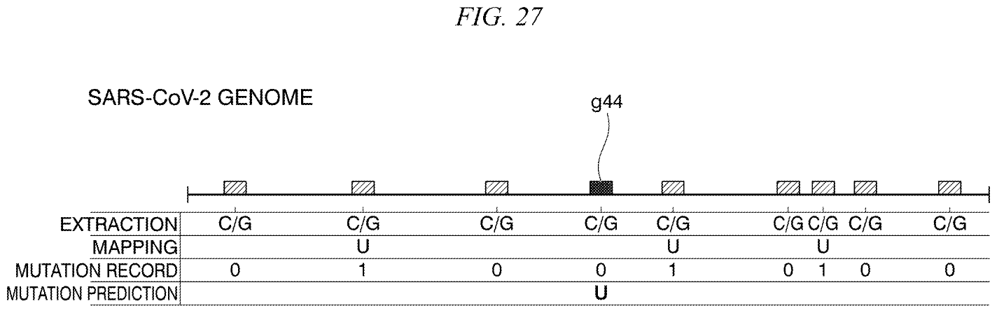

(Step S105) The prediction unit 18 maps the selected point on the location g44 on one SARS-CoV-2 genome as shown in FIG. 27 and displays the mapping image on the image display device 3. FIG. 27 is a figure showing mutation records and mutation prediction.

(Step S106) When the prediction unit 18 detects that the extraction site is selected through the operation of the operation unit 20 on the displayed image (FIG. 27), the prediction unit 18 displays the corresponding location in FIG. 26 (backcasting function). Here, the prediction unit 18 may display all of FIG. 25 to FIG. 27 on one screen or may display at least one by switching. The embodiment enables selection and mapping in both directions in FIG. 26 and FIG. 27, for example.

The processing procedures shown in FIG. 24 are examples, and the invention is not limited thereto.

As described above, as a result of the comprehensive analysis of 7800 gene sequences of the genomes of the novel coronavirus of the world, the gene mutations of the virus were found to have characteristics. The characteristics found are: 1) there are many uracil (U) mutations; 2) there are many mutations from cytosine (C) to uracil (U); 3) RNA editing enzymes are involved in gene mutations; and 4) there are characteristic sequences of one base to three bases before and after uracil mutations. Moreover, because coronaviruses have RNA-proofreading enzymes, it was speculated that mutations are limited to point mutations and that mutations by RNA editing enzymes are evident. As a result, in the embodiment, by focusing on RNA editing enzymes and searching the viral genomes based on the characteristic sequences of several bases before and after gene mutations of the virus, prediction of a site which may be mutated in the future and the substituting base has been enabled. That is, according to the embodiment, a mutation of the novel coronavirus which may occur in the further can be predicted.

In the embodiment, the viral genomes were searched based on the characteristic sequences of several bases before and after gene mutations of the virus, and machine learning and prediction of a mutation are conducted using the past mutations (from C or G to U) as the teaching data.

As a result, in the embodiment, prediction of a viral mutation with an accuracy rate of 60 to 70% has been enabled. The percentage of correct answers, however, is the percentage of correct answers including not only mutations by RNA editing enzymes but also spontaneous mutations, and thus it can be easily thought that the percentage of correct answers of prediction of a mutation by an RNA editing enzyme is higher when spontaneous mutations and mutations by RNA editing enzymes only are distinguished. Here, the AUC (Area Under the Curve) score was used as the percentage of correct answers above. Calculation of the AUC scores and the like will be described below.

Therefore, according to the embodiment, when a viral mutation can be predicted in advance before the mutation occurs, a diagnostic kit can be prepared in advance for diagnosing viral infection. According to the embodiment, the invention enables development of an ultra-early diagnostic kit. Moreover, according to the embodiment, not only provision of a diagnostic kit, but also assessment of the effects of a vaccine, assessment of the effects of a viral antibody medicine and certification and revocation of an immunity passport are enabled. Additionally, according to the embodiment, because selection of a candidate therapeutic agent is also enabled, ultra-early treatment is also enabled.

[Verification Results]

An example of the results of verification of the learning and the prediction above is explained below.

It was found that the count of U in the viral genome increases through point mutations. Because enhancement of inflammation was expected through the increased U count, it was examined whether the inflammatory cytokine production would change or not. For cell stimulation assay, four different sequences, namely EPI_ISL 419308, EPI_ISL 415644, EPI_ISL 418420 and EPI_ISL 419846, were selected from SARS-CoV-2 variants. The mutated sequences were detected in Japan, Georgia, France and Australia, respectively.

From the full length of single strand RNA (ssRNA) of each of the four mutants, the operator extracted one region in which a mutation to U was observed and synthesized the region.

The ssRNA sequences obtained from the different variants were as follows: variant-1 (5′-AUUUAUUGUUCUUUUACCC-3′; at 2946-2965 region in EPI_ISL 419308); variant-2 (5′-AUUUAUUGUUCUUUUUCUUUUACCC-3′; at 11041-11060 region in EPI_ISL 415644); variant-3 (5′-UUUCUACAGUGUCCCACUU-3′; at 14392-14411 region in EPI_ISL 418420) and variant-4 (5′-AAACCUUUGAGAGAGUU-3′; at 22946-22965 region in EPI_ISL 419846).

The same regions in a reference sequence (MN908947) were used as controls for the mutated SARS-CoV-2 sequences. The reference sequences corresponding to the respective four different mutants were Wuhan-1 (5′-AUGUAAUGUUCUCCC-3′; at 3023-3042 region), Wuhan-2 (5′-UCUCUAUGUCUCUCUCCUCCC-3′; at 11066-11085 region), Wuhan-3 (5′-UCUCUAUCAGUCCCUCCCUCCUCUCU-3′; at 14390-14409 region and 11066-11085 region), Wuhan-3 (5′-UCUCUACCUACGUGUCCCCUCU-3′; at 14390-14409 region) and Wuhan-4 (5f-AAACCCUACUUUGUAGAGAGUAUAU-3′; at 22946-22965 region).

For inducing TLR7-mediated cytokine production, a sequence containing no U (5′-GACAGAGAGAGAACAAG-3′) was used as a negative control. For verification, ssRNAs synthesized by Nihon Gene Research Laboratories Inc. (Sendai, Miyagi) were used.

A human monocytic leukemia cell line, THP-1, was maintained in RPMI-1640 medium supplemented with 10% FCS, 55 mM 2-mercaptoethanol, 100 mM non-essential amino acids (NEAAs), 1 mM pyruvic acid and 20 mM ml-1 penicillin and streptomycin.

4×10∧5 cells were cultured in 150 μl of RPMI using a 96-well flat bottom plate. A pseudo-infection model was performed according to Yan Li et al.

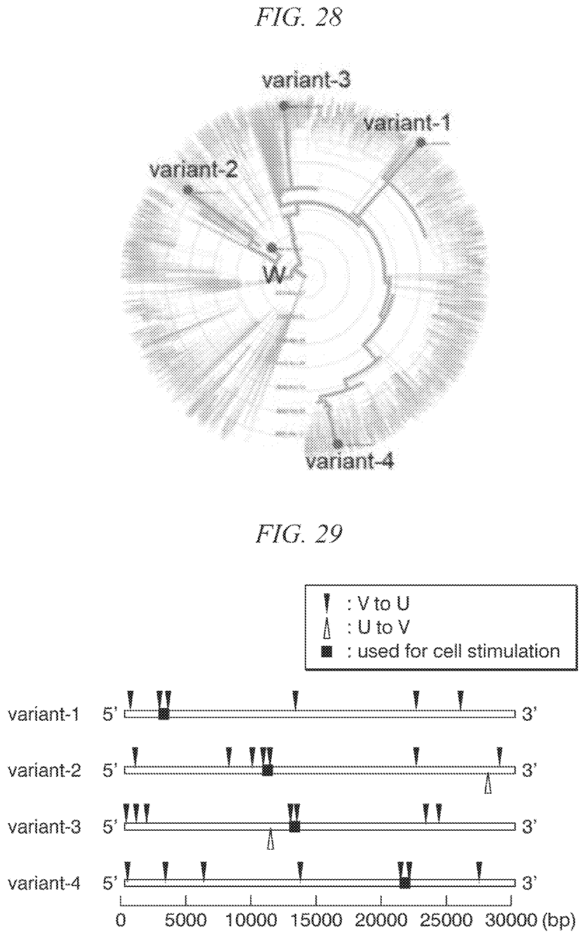

The present inventor and the like collected gene sequences from GISAID based on the initially reported Wuhan type (W) and created the phylogenetic tree in FIG. 28. FIG. 28 is a figure showing a phylogenetic tree. For verification, to examine the frequency of point mutations to U in the full length of each RNA sequence, the four different sequences below were selected from the SARS-CoV-2 mutants. The four sequences are derived from the first variant (variant-1, Japanese type), the second variant (variant-2, Georgian type), the third variant (variant-3, French type) and the fourth variant (variant-4, Australian type). In FIG. 28, W indicates the original SARS-CoV-2 sequence reported in Wuhan.

FIG. 29 is a figure showing the mutation sites on the genomes of the four selected mutated forms and the locations of the RNA sequences used for the pseudo-infection model. In FIG. 29, the horizontal direction is (bp). The inverted triangles are V-to-U (V is any base except for U), and the triangles are U-to-V. The squares show the ssRNA sequences used for cell stimulation. As shown in FIG. 29, the U counts in the full-length ssRNAs of the SARS-CoV-2 mutants were significantly higher than that of the original isolate. Moreover, as shown in FIG. 29, the frequencies of point mutations to U were much higher than the frequencies from U to A, G or C. In this manner, the ability of the full-length mutated ssRNAs to induce inflammatory cytokines is much higher than that of the original isolate. The results suggest that the mutations in the SARS-CoV-2 genes may be one of the mechanisms causing facilitation of inflammatory activation.

Some studies conducted so far have shown that U-rich ssRNA stimulates innate immune cells through TLR7 signals and produces inflammatory cytokines. Thus, it was hypothesized that many U residues derived from point mutations promote the induction of inflammatory cytokines by human macrophages.

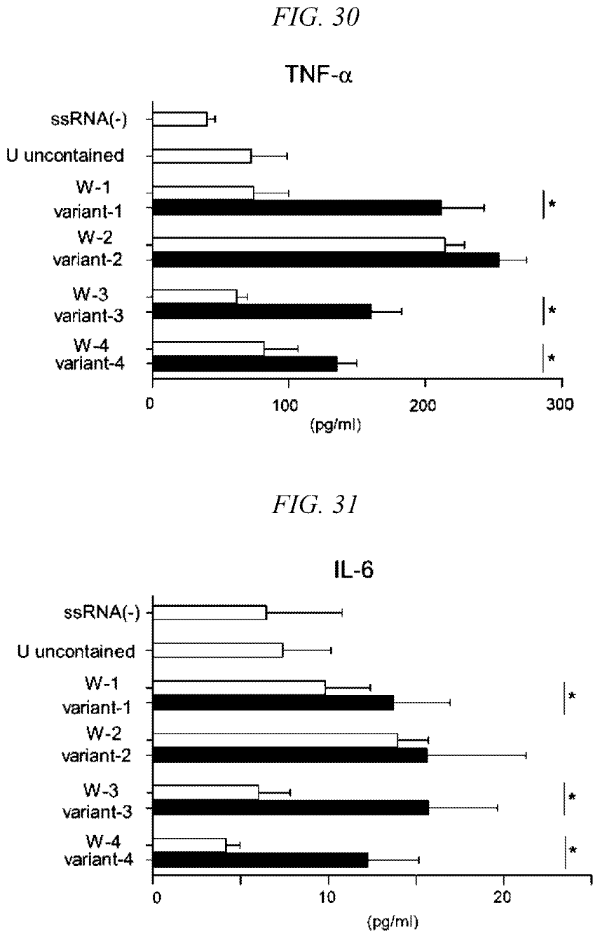

To verify the hypothesis, the production of TNF-α and IL (interleukin)-6 in a human monocyte/macrophage cell line, THP-1, which was stimulated with U-rich regions of the SARS-CoV-2 mutants was analyzed. FIG. 30 is a figure showing the TNF-α production induced by the ssRNAs. To measure human TNF-α, the cells were cultured in the presence of PMA (0.2 ng/ml, Sigma Aldrich, St. Louis, Mo., USA) and stimulated with 160 (μmol) of the ssRNAs using DOTAP (10 μg, Roche Diagnostics, Mannheim, Germany). To detect the cytokines, human TNF-α and IL-6 were measured in the culture supernatants using OptEIA set (BD Bioscience, San Diego, Calif. USA). The production of TNF-α was measured after 18-hour stimulation.

In FIG. 30 and FIG. 31, for example, W-1 is the initial Wuhan type, and variant-1 is a mutated form.

FIG. 31 is a figure showing the IL-6 production induced by the ssRNAs. To measure human IL-6, the cells were cultured in the presence of PMA (50 ng/ml) and stimulated with 480 (μmol) of the ssRNAs using DOTAP (15 μg). The production of IL-6 was measured after 48-hour stimulation.

The values are averages ±SD (n=6). The data are representative of two independent experiments with similar results.

The Fisher's exact test was performed by a one-tailed test using scipy 1.4.1 of the Python 3 base package. Mann-Whitney U test was performed using Prism 8 software (GraphPad Software, San Diego, Calif.). A value of P<0.05 indicates a significance.

As shown in FIG. 30, the ssRNA sequence lacking U residues did not upregulate the production of TNF-α as expected. The increase in the U count induced by point mutations increased the cytokine production in variants-1, 3 and 4 compared to that with the stimulation with the reference ssRNA sequence of Wuhan type.

As shown in FIG. 31, a similar tendency was observed in the production of IL-6 although the production of IL-6 was lower than that of TNF-α. The results show that point mutations to U in the SARS-CoV-2 genomes give the ability of stimulating an increase in the production of inflammatory cytokines such as TNF-α and IL-6. That is, prediction of a U mutation can also predict enhancement of inflammatory cytokine production, and thus the symptom of inflammation and severity of patients can also be distinguished.

In the embodiment, the acquisition unit acquires gene sequence data of a genome of a virus. The extraction unit extracts C (cytosine) or G (guanine) from the acquired gene sequence data of the genome and extracts contexts in which a mutation from C or G to U (uracil) occurs or has occurred.

In the embodiment, as described above, when C or G has changed to U in the base sequences of the extracted contexts, whether there is an amino acid mutation is checked. A mutation by an RNA editing enzyme directly acts on the genome RNA and induces a mutation and thus is believed to be caused regardless of the presence or absence of an amino acid mutation. However, when there is an amino acid mutation, there must be data on viruses which do not exist or genomes which do not exist because there are mutations involving the survival of the virus regardless of the cause of the mutations. Accordingly, the mutation data including amino acid mutations themselves are believed to be biased data. Thus, it is reasonable to use data without amino acid mutations for learning data.

Thus, in the embodiment, the separation unit separates sequences with an amino acid mutation as nonsynonymous substitutions and separates sequences without an amino acid mutation as synonymous substitutions. Then, the learning unit learns using the sequence data of the synonymous substitutions for learning data, and the prediction unit predicts a mutation of the virus using the learned results.

[Analysis Program]

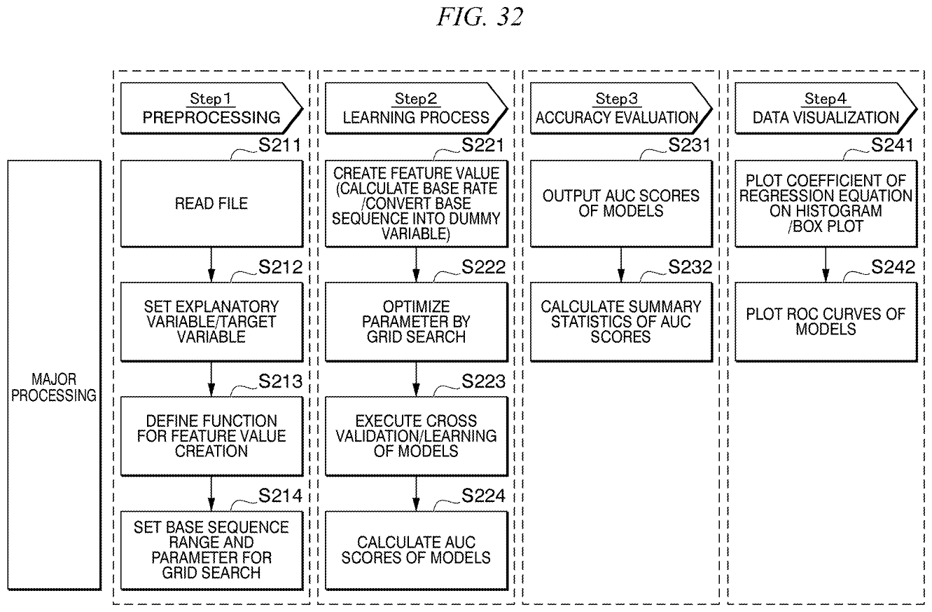

Here, an example in which the viral mutation prediction device 1 described above is achieved with an analysis program which is a software program is explained. FIG. 32 is a figure showing example processing contents and example processing procedures of the analysis program according to the embodiment. In FIG. 32, the vertical direction is major processing, and the horizontal direction is processing procedures.

In preprocessing (step S210), the analysis program reads a file as the subject of analysis (step S211), sets explanatory variables/target function (step S212), defines a function for feature value creation (step S213) and sets a base sequence range and a parameter for grid search (step S214).

Here, the target variable is the presence or absence of mutation, and the explanatory variables are two, the base sequence converted into a dummy number and the base rate. The function for feature value creation is, for example, a function which calculates the base rates (percentage of each of “A”, “G”, “C” and “T” contained in one record) using the base sequence range (for example: −3 to +3) as an argument.

In a learning process (step S220), the analysis program creates a feature value (step S221), optimizes a parameter by grid search (step S222), executes cross-validation/learning of models (step S223) and calculates the AUC scores of the models (step S224)

For creating a feature value, the base rates are calculated using the function for feature value creation, and the base sequence is converted into a dummy variable using the function for converting the variable designated as the argument into a dummy number. The ACU score is the area below the curve in the graph when a ROC (Receiver Operating Characteristic Curve) curve is drawn and is a value, for example from 0 to 1, and a value closer to 1 indicates that the discriminating ability is higher.

In accuracy evaluation (step S230), the analysis program outputs the AUC scores of the models (step S231) and calculates the summary statistics of the AUC scores (step S232).

In data visualization (step S240), the analysis program shows the coefficient of a regression equation on a histogram and plots on a box plot (step S241) and plots the ROC curves of the models (step S242).

[Analysis of Optimization of Hyperparameters of Models]

Next, example results of the analysis of optimization of hyperparameters of models are explained. In the analysis, grid search of the hyperparameter of each model was conducted for each base sequence range, and an optimized value was calculated.

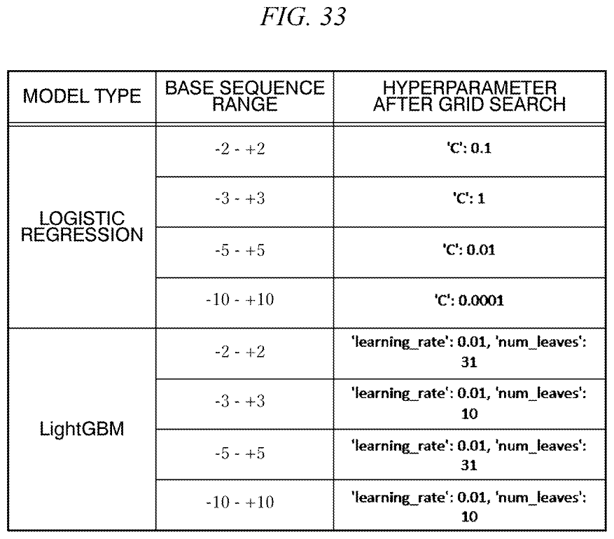

FIG. 33 is a figure showing example hyperparameter values of models which were optimized by grid search for each base sequence range. In the example of FIG. 32, the models are logistic regression and LightGBM, which is a method combining a decision tree and gradient boosting. The analysis conditions of FIG. 33 are the number of cross-validation rounds of five. The base sequence ranges are −2 to +2, −3 to +3, −5 to +5 and −10 to +10. The hyperparameter used for verification of logistic regression is C: [0.0001, 0.001, 0.01, 0.1, 1, 10, 100, 1000], and the hyperparameters used for verification of LightGBM are num_leaves: [10, 31, 64] and learning_rate: [0.01, 0.1, 1].

As shown in FIG. 33, there is a tendency towards increased strength of regularization in logistic regression as the base sequence range expands. Moreover, the hyperparameter “learning_rate” of LightGBM was constant and was 0.01.

[Comparison of Correlation Coefficients of Logistic Regression of Base Sequence Ranges]

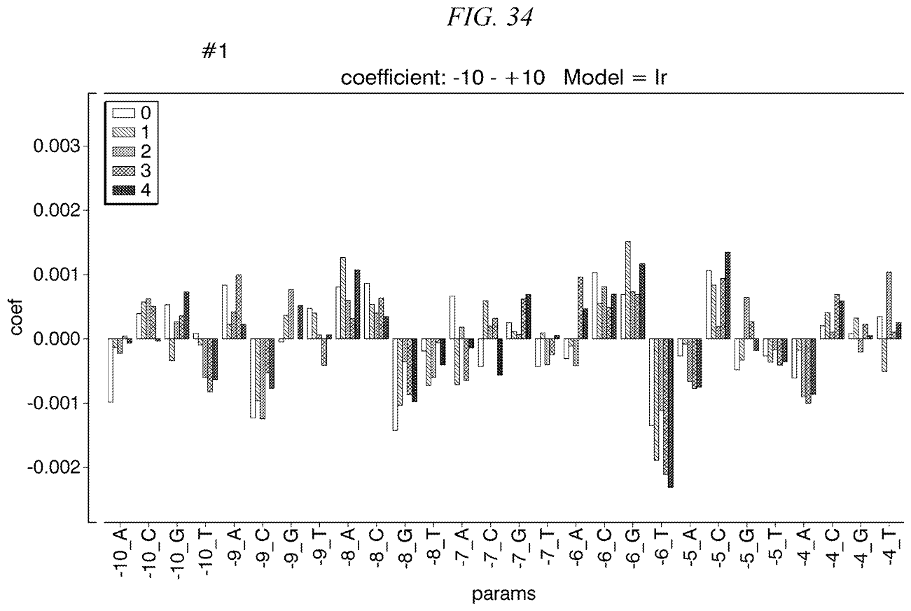

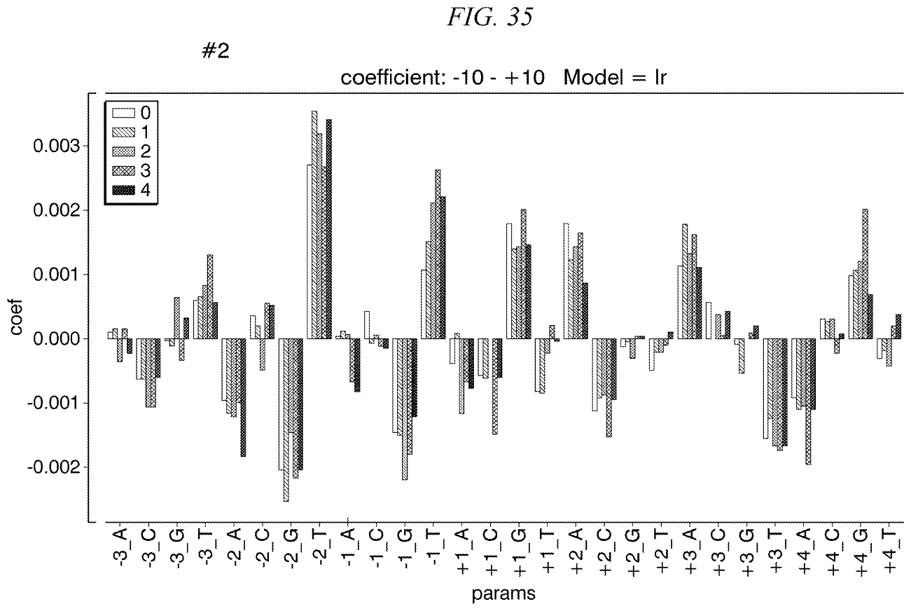

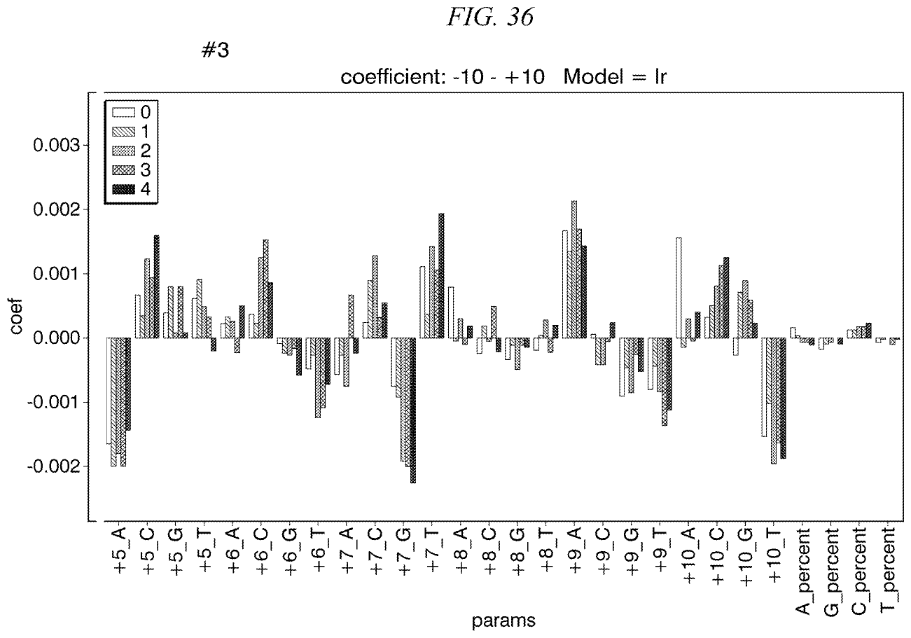

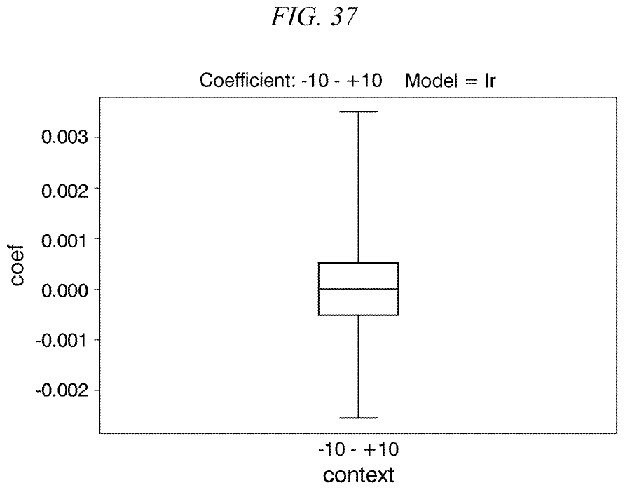

Next, as an example of the results of comparison of the correlation coefficients of logistic regression for the base sequence ranges of −2 to +2, −3 to +3, −5 to +5 and −10 to +10, the results for the base sequence range of −10 to +10 are shown in FIG. 34 to FIG. 37. In the analysis, the correlation coefficients of logistic regression of the variables of the base sequence ranges were plotted and compared among the groups. Here, FIG. 34 to FIG. 36 show the analysis results of A, C, G, T, A_percent, G_percent, C_percent and T_percent. Here, A_percent, G_percent, C_percent and T_percent are the proportions of the bases per one record. Moreover, in FIG. 34 to FIG. 36, 0 to 4 are the coefficients per cross round, and for example, the bar of 0 is the coefficient of the first cross-validation. In FIG. 34 to FIG. 36, the horizontal axis is the parameter, and the vertical axis is the proportion. In FIG. 37, the vertical axis is the proportion.

FIG. 34 to FIG. 36 are figures showing the coefficients of a regression equation for the base sequence range of −10 to +10 on a histogram. FIG. 37 is a figure showing a box plot of a histogram of the coefficients of a regression equation for the base sequence range of −10 to +10. As shown in FIG. 34 to FIG. 36, the values of −6T, −2G, −2T, −1G, −1T, +5A and the like were large for the base sequence range of −10 to +10.

Moreover, the values of −2T and +1G were large for the base sequence range of −2 to 2. The values of −2T, −1G and +1G were large for the base sequence range of −3 to 3. The values of −2T, −1G, −1T, +1G and the like were large for the base sequence range of −5 to 5.

Here, such correlation coefficients were used for visualizing the weights of the bases described below.

[Summary Statistics of AUC Scores of Models]

Next, example results of analysis of the summary statistics of the AUC scores of models are explained. In the analysis, the summary statistics of AUC scores of each learning algorithm were calculated.

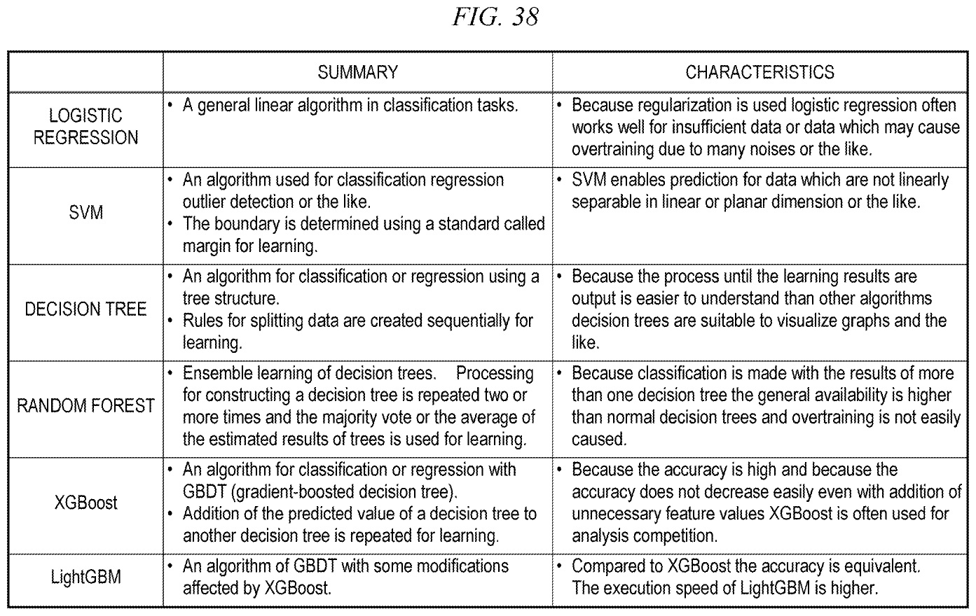

FIG. 38 is a figure showing the summary and the characteristics of the compared learning models. As shown in FIG. 38, the models are logistic regression, SVM (Support Vector Machine), a decision tree, a random forest, XGBoost and Light GBM.

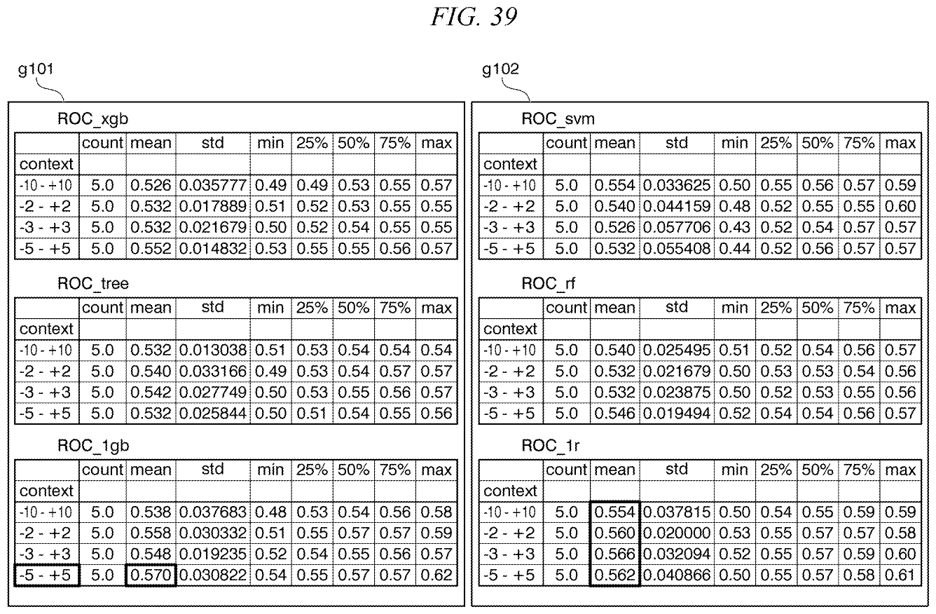

FIG. 39 is a figure showing example results of analysis of the summary statistics of AUC scores of the models. FIG. 39 shows the summary statistics using the correlation coefficients of logistic regression for the base sequence ranges of −2 to +2, −3 to +3, −5 to +5 and −10 to 10 where the figures were rounded down to the second decimal place. The image g101 in FIG. 39 shows ROC of XDBoost (ROC_xgt), ROC of a decision tree (ROC tree) and ROC of LightGBM (ROC_lgb). The image g102 shows ROC of SVM (ROC_svm), ROC of a random forest (ROC_tf) and ROC of logistic regression (ROC_lr). In FIG. 39, because the data were split into five by cross-validation, the count of each summary statistic is 5. The mean is the score (synonymous with the percentage of correct answers), and the std is the standard deviation. The min is the minimum value, and the max is the maximum value.

As shown in FIG. 39, the scores for the base sequence range of −10 to +10, the base sequence range of −2 to +2, the base sequence range of −3 to +3 and the base sequence range of −5 to +5 were 55.4%, 56.0%, 56.6% and 56.2%, respectively. Thus, the scores of logistic regression were high as a whole. The scores around 52 to 57% were obtained for the other models.

Next, the AUC scores before processing and after processing of a case using logistic regression as the model are explained.

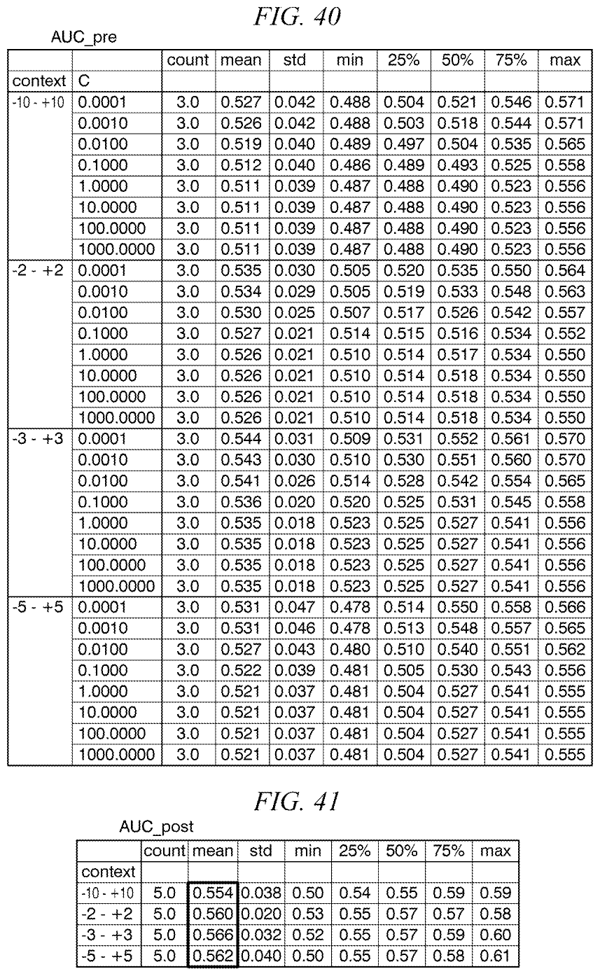

FIG. 40 is a figure showing example AUC scores before processing. FIG. 41 is a figure showing example AUC scores after processing. FIG. 40 and FIG. 41 show the summary statistics using correlation coefficients of logistic regression for the base sequence ranges of −2 to +2, −3 to +3, −5 to +5 and −10 to +10 where the figures were rounded down to the second decimal place. Moreover, in FIG. 40 and FIG. 41, the AUC scores after processing were calculated after deleting the variables shown below which did not exist in the data before processing, for comparison. The deleted variables (hyperparameters) are A_percent, G_percent, C_percent and T_percent.

As shown in FIG. 40 and FIG. 41, the AUC scores before processing of the case using logistic regression were about 51 to around 54%, but the AUC scores after processing increased to about 56 to around 57%.

[ROC Curves of Models]

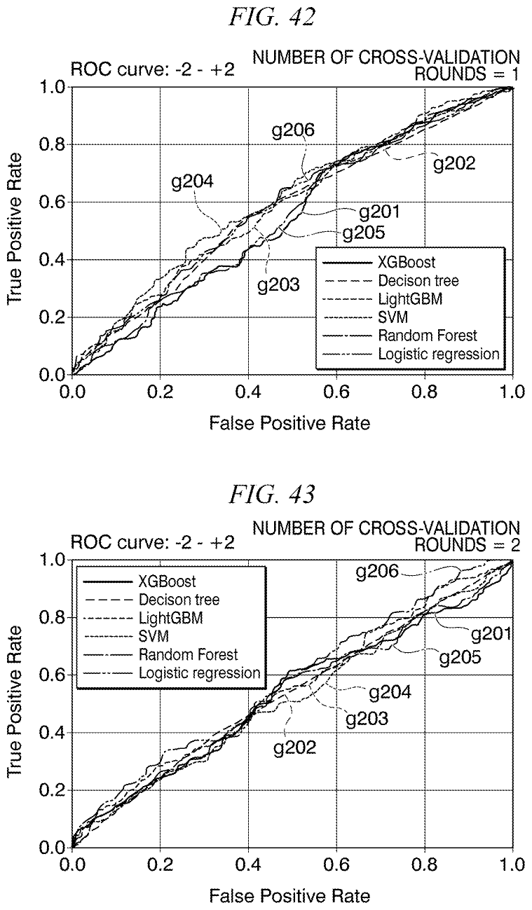

Next, example results of analysis using the ROC curves of models are explained. In the analysis, the ROC curves of the learning algorithms were plotted for the base sequence ranges of −2 to +2, −3 to +3, −5 to +5 and −10 to +10 and compared among the models. As an example of the comparison results, example comparison results for the base sequence range of −2 to +2 are shown in FIG. 42 and FIG. 43. FIG. 42 is a figure showing the ROC curves of the models for the base sequence range of −2 to +2 and the first cross-validation round. FIG. 43 is a figure showing the ROC curves of the models for the base sequence range of −2 to +2 and the second cross-validation round. In FIG. 42 and FIG. 43, the horizontal axis is the false positive rate (1.0=100%), and the vertical axis is the score (1.0=100%). The algorithms used are logistic regression, SVM, a decision tree, a random forest, XGBoost and Light GBM shown in FIG. 38. In FIG. 42 and FIG. 43, the line g201, the line g202, the line g203, the line g205, the line g205 and the line g206 are the ROC curves of XGBoost, a decision tree, Light GBM, SVM, a random forest and logistic regression, respectively.

From FIG. 42, FIG. 43 and the results of the base sequence ranges of −3 to +3, −5 to +5 and −10 to +10, comparable results were obtained for all the used models, but the difference in the ROC areas of SVM was large depending on the base sequence range compared to the other models.

[Actual Example of Machine Learning]

In order to conduct the analysis described above or the like, a program achieving the features of the viral mutation prediction device 1 has the following features.
I. A first function for reading a file as the subject of analysis and deleting the records of “1” which are not used for analysis.
II. Executing a second function for calculating base rates, calculating the base rates of the data read in I and storing in a new variable.
III. Converting the variables (for example, rows C to V of the file) of the base sequences of the data read in I into dummy variables using a third function.
IV. Executing grid search using a fourth function and optimizing the parameters of the models (FIG. 33).
V. Executing 5-fold cross-validation using a fifth function.
VI. Setting the variables in II and III as the explanatory variables and the presence or absence of a mutation (for example, row B of the file) of the data read in I as the target variable in a first method and executing learning of the models. In the first method, by setting the test data of the subjects of classification for a first argument and the correct answer of the classified results for a second argument, machine learning is conducted.
VII. Calculating the AUC scores of the models using a sixth function based on the learning results in VI.
VIII. Calculating the summary statistics of the AUC scores of the models by a second method for extracting statistical information (for example, FIG. 38 to FIG. 43).
IX. Plotting the coefficients of logistic regression using a third method (for example, FIG. 34 to FIG. 36). The third method is a method which uses the average of the given vectors (sequences constituted by values) as the height and outputs the confidence interval as the error bar.
X. Plotting the coefficient on a box plot using the third method (for example, FIG. 37).
XI. Plotting the ROC curves of the models using a fourth method for plotting (for example, FIG. 42 and FIG. 43).

The features, the functions and the methods of I to XI described above are examples, and the invention is not limited thereto.

[Splitting Method of Learning Data and Method for Measuring Generalization Performance]

Next, the method for splitting learning data and the method for measuring generalization performance are explained.

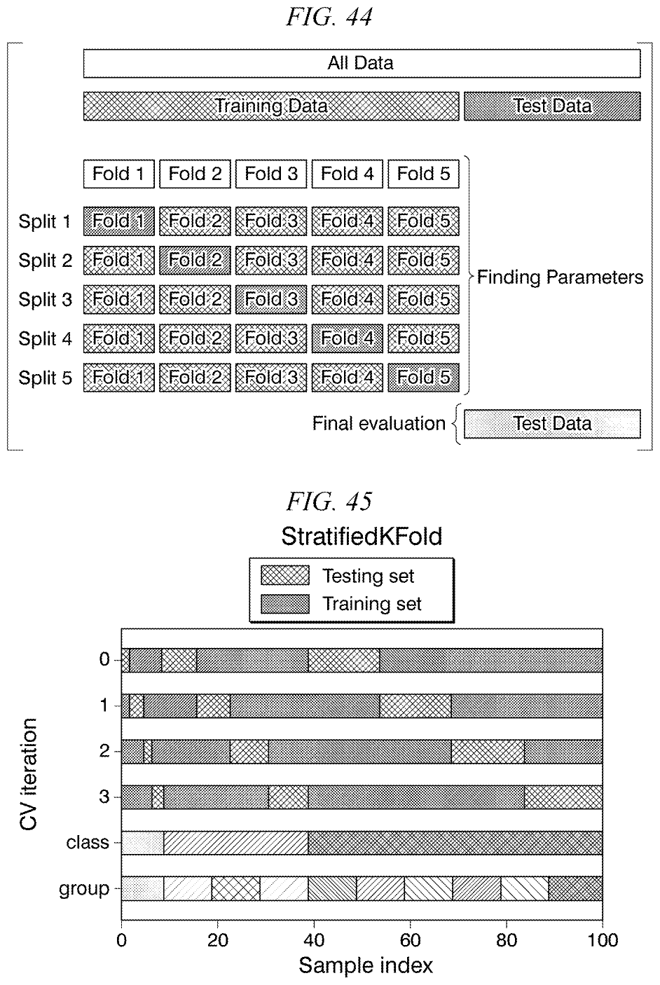

FIG. 44 is a figure showing an example method for splitting learning data by five cross-validation rounds.

How the learning data and the test data are split is a very important issue. Thus, in the embodiment, the training data and the test data were split as shown in FIG. 44, and learning was conducted while switching the training data and the test data for each cross round.

FIG. 45 is a figure for explaining the method for measuring generalization performance.

In the embodiment, as shown in FIG. 45, StratifiedKFold was used as the method for measuring the generalization performance. In the processing, the data are split into training and test data while maintaining the ratio of the distribution.

The examples shown in FIG. 44 and FIG. 45 are examples, and the invention is not limited thereto.

[G-to-U, G-to-A, A-to-G and U-to-C]

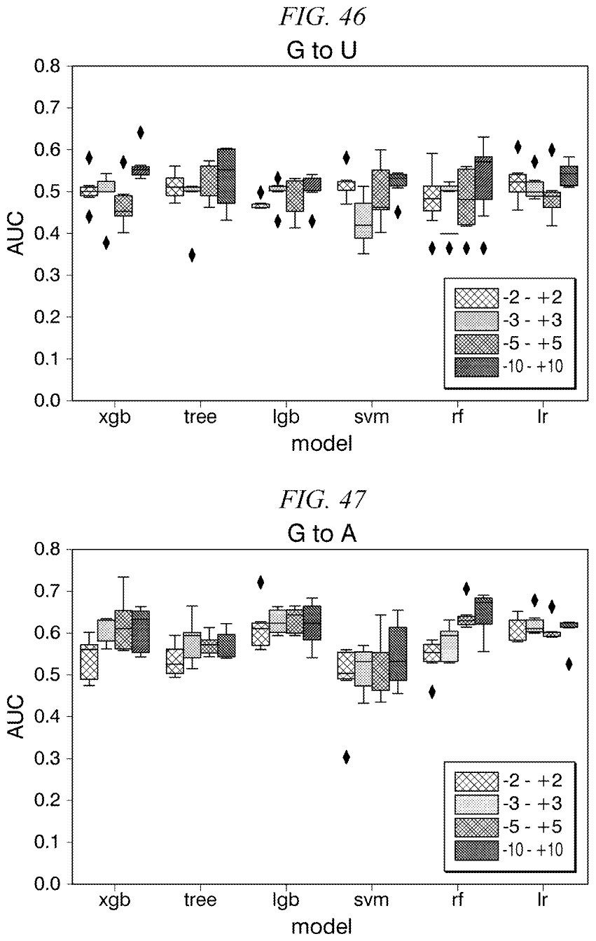

An example in which contexts in which a mutation from C (cytosine) or G (guanine) to U (uracil) occurs or has occurred are extracted has been explained above, but the invention is not limited thereto. Example learning results of other mutation examples are shown below in FIG. 46 to FIG. 49. In this case, contexts in which a mutation from G to U, from G to A, from A to G or from U to C (or from T (thymine) to C) occurs or has occurred are extracted. In learning and estimation, U (uracil) is extracted from those described as RNA, and T (thymine) is extracted from those described as DNA.

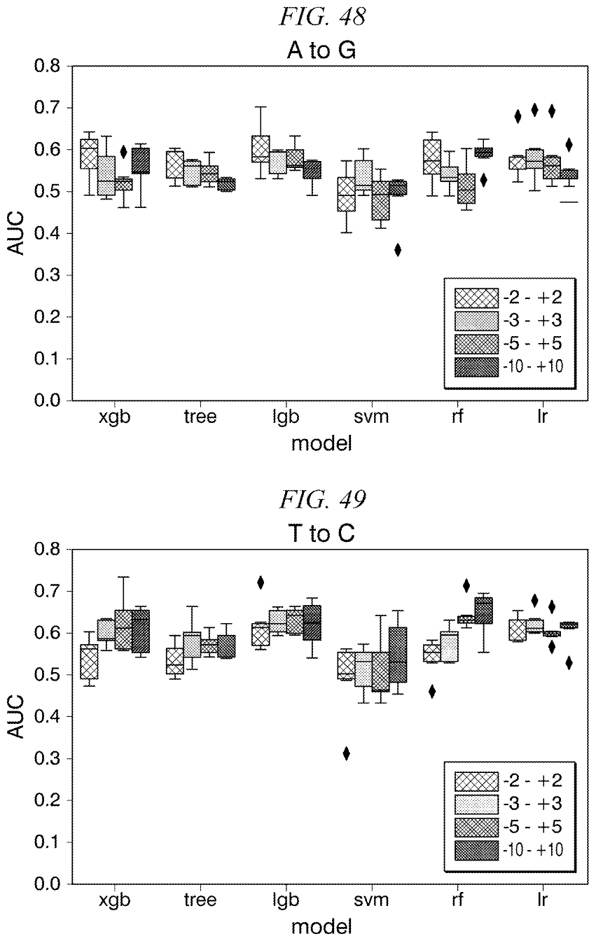

FIG. 46 is a box plot showing the base sequence ranges and the learning models for mutations from G to U. FIG. 47 is a box plot showing the base sequence ranges and the learning models for mutations from G to A. FIG. 48 is a box plot showing the base sequence ranges and the learning models for mutations from A to G. FIG. 49 is a box plot showing the base sequence ranges and the learning models for mutations from U to C (or from T (thymine) to C). FIG. 49 cites T-to-C in terms of DNA, but the mutation is U to C in terms of RNA.

In the explanation below, xgb indicates XGBoost, and Tree indicates a decision tree. Lab indicates Light GBM, and Svm indicates SVM. rf indicates a random forest, and Lr indicates logistic regression.

For mutations from G to U, for example, the average percentage of correct answers for the base sequence range of −10 to +10 of XGBoost was 56.4%, and the average of a decision tree was 53.0%. The average of Light GBM was 50.0%, and the average of SVM was 51.4%. The average of a random forest was 54.0%, and the average of logistic regression was 54.0%.

As shown in FIG. 46, for mutations from G to U, the results of the combination of the base sequence range of −10 to +10 and the model XGBoost were the best.

For mutations from G to A, for example, the average percentage of correct answers for the base sequence range of −5 to +5 of XGBoost was 62.2%, and the average of a decision tree was 57.0%. The average of Light GBM was 62.8%, and the average of SVM was 52.6%. The average of a random forest was 64.2%, and the average of logistic regression was 60.2%. Moreover, the average percentage of correct answers for the base sequence range of −10 to +10 of XGBoost was 60.6%, and the average of a decision tree was 56.6%. The average of Light GBM was 61.6%, and the average of SVM was 54.4%. The average of a random forest was 64.2%, and the average of logistic regression was 59.8%.

As shown in FIG. 47, for mutations from G to A, the results of the combination of the base sequence range of −10 to +10 or −5 to +5 and the model random forest were the best.

For mutations from A to G, for example, the average percentage of correct answers for the base sequence range of −2 to +2 of XGBoost was 58.0%, and the average of a decision tree was 56.4%. The average of Light GBM was 60.2%, and the average of SVM was 48.8%. The average of a random forest was 57.2%, and the average of logistic regression was 58.2%.

As shown in FIG. 48, for mutations from A to G, the results of the combination of the base sequence range of −2 to +2 and the model Light GBM were the best.

For mutations from U (or T) to C, for example, the average percentage of correct answers for the base sequence range of −5 to +5 of XGBoost was 61.0%, and the average of a decision tree was 62.4%. The average of Light GBM was 64.0%, and the average of SVM was 55.0%. The average of a random forest was 62.4%, and the average of logistic regression was 62.6%.

As shown in FIG. 49, for mutations from U (or T) to C, the results of the combination of the base sequence range of −5 to +5 and Light GBM were the best.

As shown above, when the method of the embodiment is used, XGBoost, a decision tree, Light GBM, SVM, a random forest and logistic regression can be used as learning models. As a result, according to the embodiment, a point mutation can be predicted with high accuracy using the learned results.

Moreover, according to the embodiment, a point mutation can be predicted using the learned results using the method of the embodiment for mutations from G to A, mutations from A to G and mutations from T to C in addition to mutations from G to U.

The descriptions of the contexts in the explanation above and in the figures are explained.

In the present specification, a context is described with the mutation site indicated by 0, the upstream side indicated by minus (−) and the downstream side indicated by plus (+). Moreover, in the figures and the specification, plus is indicated in some cases and is not indicated in other cases (for example, “1 G” and “+1 G”), but they refer to the same context. In the figures and the specification, an underscore is between a number and an alphabet in some cases and is not in other cases, for example as in “1 G” and “+1 G” and “1G” and “+1G”, but they refer to the same context.

Moreover, regarding the base sequence ranges, for example, the range of −2 to +2 is described as “−2-+2” or “−2 to +2” in the specification and the figures.

A program for achieving all or a part of the features of the viral mutation prediction device 1 in the invention may be recorded on a recording medium which can be read by a computer, and the program recorded on the recording medium may be read by a computer system and executed to conduct all the processing or a part of the processing conducted by the viral mutation prediction device 1. For machine learning, various learning methods such as deep learning may be used, and processing may be conducted using artificial intelligence (AI: Artificial Interigence). The “computer system” here includes an OS and hardware such as a peripheral device. The “computer system” also includes a WWW system equipped with an environment for providing a homepage (or an environment for display). The “recording medium which can be read by a computer” refers to a portable medium such as a flexible disk, a magneto-optical disc, a ROM and a CD-ROM and a memory device such as a hard disk installed in the computer system. The “recording medium which can be read by a computer” also includes a medium which keeps the program for a certain period, such as a server to which the program has been transmitted through a network such as internet or a communication line such as a telephone line and volatile memory (RAM) in the computer system as a client.

The program may be transmitted from the computer system in which the program is stored in a memory device or the like, through a transmission medium or with a transmission wave in a transmission medium, to another computer system. Here, the “transmission medium” which transmits the program refers to a medium which has the function of transmitting information, such as a network (communication network) like internet or the like and a communication line like a telephone line or the like. The program may be for achieving a part of the features described above. The program may be a so-called differential file (a differential program) which can achieve the features described above when combined with a program which is already recorded on the computer system.

Although modes for carrying out the invention have been explained above using embodiments, the invention is not limited to the embodiments at all, and various changes and substitutions can be added in the scope which does not go beyond the gist of the invention.

### REFERENCE SIGNS LIST

1 viral mutation prediction device
2 DB
3 image display device
11 acquisition unit
12 memory unit
13 extraction unit
14 separation unit
15 sampling unit
16 feature value addition and selection unit
17 learning unit
18 prediction unit
19 output unit
20 operation unit
A adenine
U uracil
G guanine
C cytosine
T thymine

1. A viral mutation prediction device comprising:
an acquisition unit which acquires gene sequence data of a genome of a virus,
an extraction unit which extracts C (cytosine) or G (guanine) from the acquired gene sequence data of the genome and extracts contexts in which a mutation from C or G to U (uracil) occurs or has occurred,
a separation unit which checks whether there is an amino acid mutation when C or G has changed to U and which separates sequences with the amino acid mutation as nonsynonymous substitutions and separates sequences without the amino acid mutation as synonymous substitutions,
a learning unit which learns using the sequence data of the synonymous substitutions for learning data and
a prediction unit which predicts a mutation of the virus using the learned results.

2. A viral mutation prediction device comprising:
an acquisition unit which acquires gene sequence data of a genome of a virus,
an extraction unit which extracts C (cytosine), G (guanine), A (adenine), U (uracil) or T (thymine) from the acquired gene sequence data of the genome and extracts contexts in which a mutation from G to A, from A to G, from U to C or from T to C occurs or has occurred,
a separation unit which checks whether there is an amino acid mutation when the base sequences of the extracted contexts have changed and which separates sequences with the amino acid mutation as nonsynonymous substitutions and separates sequences without the amino acid mutation as synonymous substitutions,
a learning unit which learns using the sequence data of the synonymous substitutions for learning data and
a prediction unit which predicts a mutation of the virus using the learned results.

3. The viral mutation prediction device according to claim 1, further comprising:
a sampling unit which selects a predetermined number of synonymous substitutions from the synonymous substitutions,
wherein
the learning unit uses the sequence data of the synonymous substitutions selected by the sampling unit for learning data.

4. The viral mutation prediction device according to claim 1, further comprising:
a feature value addition and selection unit which adds a feature value that is a value characterized by selecting two bases from the four kinds of RNA bases, A (adenine), U, G and C, and that is used for learning,
wherein
the learning unit also uses the feature value for learning data.

5. The viral mutation prediction device according to claim 1, wherein
the range of the contexts is −3 to +3 or more and −10 to +10 or less.

6. The viral mutation prediction device according to claim 1, wherein
the virus is SARS-CoV-2.

7. A viral mutation prediction method implemented in a viral mutation prediction device that includes:
an acquisition unit acquires gene sequence data of a genome of a virus,
an extraction unit extracts C (cytosine) or G (guanine) from the acquired gene sequence data of the genome and extracts contexts in which a mutation from C or G to U (uracil) occurs or has occurred,
a separation unit checks whether there is an amino acid mutation when C or G has changed to U, separates sequences with the amino acid mutation as nonsynonymous substitutions and separates sequences without the amino acid mutation as synonymous substitutions,
a learning unit learns using the sequence data of the synonymous substitutions for learning data, and
a prediction unit predicts a mutation of the virus using the learned results.

8. A viral mutation prediction method implemented in a viral mutation prediction device that includes:
an acquisition unit acquires gene sequence data of a genome of a virus,
an extraction unit extracts C (cytosine), G (guanine), A (adenine), U (uracil) or T (thymine) from the acquired gene sequence data of the genome and extracts contexts in which a mutation from G to A, from A to G, from U to C or from T to C occurs or has occurred,
a separation unit checks whether there is an amino acid mutation when the base sequences of the extracted contexts have changed, separates sequences with the amino acid mutation as nonsynonymous substitutions and separates sequences without the amino acid mutation as synonymous substitutions,
a learning unit learns using the sequence data of the synonymous substitutions for learning data, and
a prediction unit predicts a mutation of the virus using the learned results.

9. A program that is executed in a viral mutation prediction device that includes:
a computing machine,
to acquire gene sequence data of a genome of a virus,
to extract C (cytosine) or G (guanine) from the acquired gene sequence data of the genome, to extract contexts in which a mutation from C or G to U (uracil) occurs or has occurred,
to check in a separation unit whether there is an amino acid mutation when C or G has changed to U, to separate sequences with the amino acid mutation as nonsynonymous substitutions, to separate sequences without the amino acid mutation as synonymous substitutions,
to learn using the sequence data of the synonymous substitutions for learning data and
to predict a mutation of the virus using the learned results.

10. A program that is executed in a viral mutation prediction device that includes:
a computing machine,
to acquire gene sequence data of a genome of a virus,
to extract C (cytosine), G (guanine), A (adenine), U (uracil) or T (thymine) from the acquired gene sequence data of the genome, to extract contexts in which a mutation from G to A, from A to G, from U to C or from T to C occurs or has occurred,
to check whether there is an amino acid mutation when the base sequences of the extracted contexts have changed, to separate sequences with the amino acid mutation as nonsynonymous substitutions, to separate sequences without the amino acid mutation as synonymous substitutions,
to learn using the sequence data of the synonymous substitutions for learning data and
to predict a mutation of the virus using the learned results.
